(19) United States
(12) Patent Application Publication
(10) Pub. No.: US 2017/0196913 A1
(43) Pub. Date: Jul. 13, 2017

(54) ENHANCEMENT OF GLUCOSE-STIMULATED INSULIN SECRETION BY CELLS THROUGH INDUCTION OF CELLULAR SENESCENCE

(71) Applicant: YISSUM RESEARCH DEVELOPMENT COMPANY OF THE HEBREW UNIVERSITY OF JERUSALEM LTD., Jerusalem (IL)

(72) Inventors: Ittai BEN-PORATH, Jerusalem (IL); Yuval DOR, Jerusalem (IL); Aharon HELMAN, Tzur-Hadassah (IL); Agnes KLOCHENDLER, Jerusalem (IL)

(21) Appl. No.: 15/401,289

(22) Filed: Jan. 9, 2017

Related U.S. Application Data
(60) Provisional application No. 62/275,836, filed on Jan. 7, 2016.

(51) Int. Cl. A61K 35/39; A61K 38/28; C12N 5/071

(52) U.S. Cl. CPC A61K 35/39; C12N 5/0676; A61K 38/28; C12N 2501/998

(57) ABSTRACT

Methods for increasing glucose induced insulin secretion in an insulin secreting cell by inducing senescence in the cell are provided. Further, methods for treating diabetes, by providing cells with increased glucose induced insulin secretion to a subject, as well as a population of modified insulin secreting cells are provided.

### CROSS REFERENCE TO RELATED APPLICATIONS

This application claims the benefit of priority to U.S. Provisional Patent Application No. 62/275,836, filed Jan. 7, 2016, the contents of which are incorporated herein by reference in their entirety.

### FIELD OF INVENTION

The present invention is directed to the field insulin secreting cells and methods of use thereof.

### BACKGROUND OF THE INVENTION

Aged tissues typically display decreased regenerative capacity and deterioration in overall function. Cellular senescence is thought to contribute to tissue aging and associated pathologies by various means, including limitation of stem cell proliferation and enhanced secretion of negatively-acting paracrine factors. Senescence is often activated in damaged cells, and senescent cells accumulate in aging tissues as well as in premalignant lesions. However, recent studies have also demonstrated roles for senescence in normal embryonic development, expanding the traditional view of its function as a stress-response program.

The tumor suppressor protein p16^{Ink4a} (also known as CDKN2A, hereafter p16), is transcriptionally activated in many settings of senescence, and is expressed during aging in multiple tissues. p16 inhibits CyclinD/Cyclin dependent kinase (CDK)4/6 complexes to activate Retinoblastoma (Rb), which induces widespread chromatin modification leading to senescence-associated reprogramming of gene expression (Salama et al., 2014, Genes and Development, 28:99-114). This results in complex phenotypic changes in cytoskeletal structure and metabolism, including enhanced protein turnover and secretion, and increased glucose uptake and oxidative phosphorylation (Kaplon et al., 2013, Nature, 498:109-112; Takebayashi et al., 2015, Aging Cell, 14(4):6879-697). While cellular senescence is often thought to represent a state of stress and/or dysfunction, the manner and mechanisms by which it affects cell functionality remain poorly understood.

Glucose tolerance deteriorates with age, reflecting reduced responsiveness of beta-cell (β-cells) to glucose stimulation and reduced sensitivity of peripheral tissues to insulin. The proliferative capacity of β-cells declines dramatically at an early age, potentially contributing to overall reduced β-cell mass and increased risk of diabetes. Expression of p16 increases in β-cells during aging, inhibiting β-cell regenerative capacity, and is therefore thought to contribute to age-associated deterioration of glucose homeostasis. Genetic polymorphisms in the CDKN2A locus are associated with type 2 diabetes (Bao et al., 2012, Molecular Biology Reports, 39:1609-1616), suggesting that differential regulation of this gene could influence glucose metabolism; yet the functional consequences of these polymorphisms are unknown. Components of the cell-cycle machinery, including CDK4, Rb, and E2F have been implicated in various aspects of glucose homeostasis, including short-term responses to glucose stimulation by β-cells and responses to insulin by peripheral tissues. However, it is not known whether the age-associated increase in p16-expression leads to senescence of β-cells, or whether such cells remain functional in glucose sensing and insulin secretion.

Diabetes types I and II are diseases or insulin production, glucose metabolism and ultimately glucose homeostasis. Treatments that can improve β-cell insulin secretion, and more generally beta-cell function, are desperately needed to fight these diseases along with other metabolic disorders.

### SUMMARY OF THE INVENTION

The present invention provides methods for enhancing insulin secretion in response to glucose in an insulin secreting cell. The invention also provides methods for treating a metabolic disease by inducing or enhancing expression of a senescence-associated marker in an insulin producing cell, as well as compositions comprising insulin secreting cells that are modified to express one or more senescence-associated markers.

The present invention is based, in part, on the surprising finding that expression of a senescence-associated marker in an insulin secreting cell (e.g., a beta cell) induces increased insulin secretion in response to glucose in those cells. The present invention is also based, in part, on the unexpected finding that induction of senescence in an insulin secreting cell can improve glucose homeostasis in an organism.

According to a first aspect, there is provided a method for enhancing insulin secretion in response to glucose in an insulin secreting cell, the method comprising:
a. providing an insulin producing cell,
b. subjecting the cell to a condition that induces or increases expression of a senescence marker, and
c. maintaining the cell in conditions such that the cell retains its identity as an insulin secreting cell,

thereby enhancing insulin secretion in an insulin secreting cell.

According to some embodiments, the condition that induces or increases expression of a senescence marker is selected from the group consisting of: expressing a senescence inducing gene within the cell, irradiating the cell, inducing oxidative stress in the cell, and inducing DNA damage in the cell.

According to some embodiments, the senescence inducing gene is selected from the group consisting of: p16-Ink4a, p14ARF, p53, p21 and Rb.

According to some embodiments, the senescence marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

According to some embodiments, the insulin secreting cell is a beta cell, and the maintaining comprises culturing in media that supports beta cell survival.

According to some embodiments, the insulin secreting cell is a cell engineered to secrete insulin.

According to some embodiments, the method further comprises confirming retention of an insulin secreting identity. According to some embodiments, the confirming comprises measuring a marker of cellular identity. According to some embodiments, the marker of cellular identity is selected from the group consisting of: Nkx6.1, Pdx1, Chga, Ins, Mafa, Math, Nkx2.2, Neurod1, Pax6, Neurog3, Slc2a2, Kcnj11, Abcc8, Ffar1, and Pcsk1.

According to some embodiments, the method further comprises confirming an increase of insulin secretion in response to glucose from the cell as compared to a control cell. According to some embodiments, the increase is at least a 10% increase in insulin secretion.

According to some embodiments, said subjecting does not induce DNA damage in the cell. According to some embodiments, said subjecting that does not induce DNA damage is expressing within the cell a gene selected from the group consisting of p16-Ink4a, p14ARF, p53, p21 and Rb.

According to some embodiments, the subject suffers from a metabolic disease. According to some embodiments, the metabolic disease is diabetes.

According to a second aspect, there is provided a method of treating a metabolic disease in a subject in need thereof, the method comprising subjecting an insulin-producing cell to a condition that induces or increases expression of a senescence marker and does not induce DNA damage, thereby treating a metabolic disease in the subject.

According to some embodiments, the condition is expressing within said cell any one of p16-Ink4a, p14ARF, p53, p21 or Rb.

According to some embodiments, the senescence marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

According to some embodiments, the subjecting is in-vitro or ex-vivo. According to some embodiments, the method further comprising administering the cells to the subject.

According to another aspect, there is provided a composition comprising a population of modified insulin secreting cells and a carrier, the modified insulin secreting cells express one or more senescence-associated marker.

According to some embodiments, the senescence-associated marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee

FIGS. 1A-P. p16 Induces Cell-Cycle Arrest and Senescence in Pancreatic β-Cells. (A) Dot Plots of FACS analyses of dissociated islets of p16-expressing (Insulin-rtTA/tet-p16) and control (Insulin-rtTA) mice, co-stained for insulin and transgenic human p16. 3-4 week-old mice were treated with tetracycline for 10-14 days (all panels). (B) Micrograph of β-cell specific expression of transgenic human p16 (red) in pancreatic islets (dotted line) of p16-induced and control sibling mice. Ki67-expressing β-cells (green) in induced mice are p16-negative (yellow arrows). (C) Dot Plots of FACS analyses of β-cells from p16-expressing and control mice, co-stained for p16 and Ki67; plots display only insulin-expressing islet cells (see FIG. 2). Cells were pooled from 3 mice in each group. The experiment was repeated 3 times. (D) Histogram of FACS analysis of SA-βGal activity, using the C₁₂FDG fluorescent substrate, in live dissociated islet cells of p16-expressing mice (red), and control mice (blue). Cells were pooled from 5 mice in each group. The experiment was repeated twice. (E) Histogram of FACS analysis of expression of the lysosomal marker Lamp2a in insulin-positive cells of p16-expressing and control mice. (F) Images of islets stained for p16 (red), insulin (green) and E-cadherin (white), analyzed for cell area measurement. (G) Bar graphs of mean cross-section area of p16-expressing and control β-cells, calculated based on image analysis of stained islets. >100 cells per mouse from 6 mice were scored in each group. Values shown are for p16-positive β-cells in p16-induced mice versus β-cells in control mice. **—P<0.005 (H) Forward scatter (FSC-A) FACS histograms of p16-expressing and control β-cells, indicating relative cell volume. Shown are Insulin⁺/p16⁺ cells from Insulin-rtTA/tet-p16 mice, and Insulin⁺/GFP⁺ cells from Insulin-rtTA/tet-GFP sibling mice, following transgene induction. Cells were pooled from 5 mice in each group. The results were confirmed in 5 independent experiments. (I) Representative images of control and p16-expressing islets stained for Pdx1 (blue) and phopho-S6 (pS6, green). Note increased levels within p16-expressing islets. (J) Histogram of FACS analysis of pS6 expression in p16-expressing and control β-cells. Shown are Insulin⁺/p16⁺ cells from Insulin-rtTA/tet-p16 mice, and Insulin⁺ cells from Insulin-rtTA sibling mice. (K) Bar graphs showing gene sets associated with senescence or with cell proliferation that were preferentially upregulated or downregulated in p16 expressing β-cells. Values indicate −Log₁₀ P value of significance. β-cells were sorted based on GFP expression from three Ins-rtTA/tet-GFP/tet-p16 mice and two control Ins-rtTA/tet-GFP mice following tet treatment. (L) Bar graphs of representative cell-cycle genes preferentially repressed in p16 expressing β-cells. Graph shows the mean expression level in p16-expressing β-cells (grey columns) relative to control GFP-expressing β-cells (white columns) as derived from RNAseq. (M) Bar graphs of representative senescence-associated genes upregulated in p16 expressing β-cells. (N) Bar graphs of gene sets associated with β-cell function or differentiation that were preferentially upregulated in p16 expressing β-cells. (O) Bar graphs of representative upregulated genes shown to be associated with β-cell maturation from birth to adulthood. (P) Bar graphs of representative upregulated Polycomb targets, HNF1 targets and nervous system genes associated with β-cell differentiation. Error bars in panels 1-p indicate S.E.M, P<0.05 for all changed genes.

FIGS. 2A-D. FACS analysis and sorting. (A) Dot plots showing the gating scheme for representative FACS analyses of control (Insulin-rtTA) and p16-expressing (Insulin-rtTA/tet-p16) mice. (B) Dot plots showing the gating scheme for human islet cells stained for Insulin and p16. (C) Dot plots depicting isotype control stain of mouse and human islet cells. (D) Dot plots depicting sorting of GFP-expressing dissociated islet cells from control Insulin-rtTA/tet-GFP mice (GFP only) and Insulin-rtTA/tet-p16/tet-GFP mice (co-expressing GFP and p16), after 10 days of induction. Cells in the indicated gate were sorted for RNAseq analysis.

FIGS. 3A-D. Levels of p53, p53 targets, and CDK inhibitors in p16-expressing β-cells. Representative images of control and p16-expressing islets stained for (A) p53 (brown), or (B) p21 (green). (C) Bar graphs showing relative mRNA levels of p53 transcriptional targets in GFP (white) and p16-expressing (grey) β-cells. (D) Bar graphs showing relative mRNA levels of CDK inhibitors in GFP and p16-expressing β-cells. No transcripts of p19^{ARF} were detected in either control or p16-expressing cells.

FIGS. 4A-D. Expression of markers and regulators of β-cell differentiation. Representative images of control and p16-expressing islets co-stained for p16 (red) and (A) Nkx6.1 (green), (B) Pdx1 (green), and (C) Chromogranin (green). (D) Bar graphs depicting relative mRNA levels of central makers and regulators of β-cell differentiation in GFP (white) and p16-expressing (grey) β-cells.

FIGS. 5A-K. p16 expression increases glucose-stimulated insulin secretion. (A) Scatter plot of insulin levels secreted by islets of control Insulin-rtTA mice (left) and Insulin-rtTA/tet-p16 mice (right) after 10 days of p16 induction. Islets were incubated in low glucose (2.8 mM) and then stimulated with high glucose (16.7 mM) for one hour. Dots indicate individual mice, 8 per group, mean of 3 replicates per mouse, assayed in two independent experiments. Secreted insulin levels in sample media were measured and normalized to the insulin content of each sample following islet lysis. Values are presented relative to the mean secretion level of control islets at high-glucose, defined as 1. Error bars indicate S.D. The experiment was repeated four times. (B) Bar graphs of insulin levels secreted per β-cell by islets of control and p16-expressing mice, at low glucose and high glucose. After incubation with glucose and media collection, islets were dissociated and stained for insulin, and β-cell numbers were scored by FACS. Insulin concentrations in media were measured by ELISA and normalized to β-cell number. Values are presented relative to the mean secretion level of control islets at high-glucose, defined as 1. Each sample contained islets pooled from 3 individual mice, assayed in 5 replicates per group. Error bars indicate S.D. (C) Bar graph of insulin levels secreted by islets of p16-expressing and control mice after 2 months of tet treatment. Samples contained islets pooled from 3 individual mice per genotype, 5 replicates per group. Error bars indicate S.D. Values were normalized to islet insulin content as in FIG. 5A, and are presented relative to the mean secretion value of the control sample at high glucose defined as 1. Error bars indicate S.D. (D) Bar graph of insulin content in equal numbers of (3-cells sorted based on GFP expression, from control (Insulin-rtTA/tet-GFP, white) or p16 (Ins-rtTA/tet-GFP/tet-p16 mice, grey) mice. Values are presented relative to control. Samples contained islets pooled from 2 individual mice per genotype. (E) Line graph of representative glucose tolerance test of wild type mice, Pdx1-tTA mice and Pdx1-tTA/tet-p16 mice following p16 activation for 2 weeks. Error bars indicate S.E.M. The experiment was repeated 3 times. (F) Bar graphs showing serum insulin levels after overnight fasting (fast) and 10 minutes after glucose injection (glucose), in Pdx1-tTA mice (white) and Pdx1-tTA/tet-p16 mice (grey). Error bars indicate S.E.M. (G) FSC histogram of Insulin⁺ cells from mice in FIG. 5E, pooled. (H) Line graph showing representative insulin tolerance test in wild type, Pdx1-tTA and Pdx1-tTA/tet-p16 mice following p16 activation for 10 days. Error bars indicate S.E.M. (I) Line graphs of representative glucose tolerance test in wild type, Pdx1-tTA and Pdx1-tTA/tet-p16 mice following p16 activation for 2 months. Error bars indicate S.E.M. (J) Line graphs of representative glucose tolerance test in the same mice following p16 activation for 5 months. (K) Bar graphs of islet area of Pdx1-tTA mice (white) and Pdx1-tTA/tet-p16 mice (grey), following 2 weeks, 2 months or 5 months of p16 induction. Values indicate the mean percentage of pancreatic sections positively stained for insulin, as measured by image analysis. Representative sections are shown in FIG. 8B. Error bars indicate S.E.M. In all panels *—P<0.05; **—P<0.005; ***—P<0.0005; ns—non-significant.

FIGS. 6A-B. Insulin secretion following activation of p16 in β-cells—additional data. (A) Bar graphs of insulin levels secreted by islets of control (Insulin-rtTA, white) and p16-expressing (Insulin-rtTA/tet-p16, grey) mice incubated in low glucose (2.8 mM) or in low glucose followed by high glucose (16.7 mM) stimulation for 1 hr. Experiment includes islets pooled from 3 mice per genotype, 5 replicates per group. Error bars indicate S.D. Secreted insulin levels were normalized to the total protein content of each sample following islet lysis. Values are presented relative to the secretion level of control islets at high-glucose, defined as 1. (B) Bar graphs of insulin levels secreted by islets of control and p16-expressing mice following induction at 6 weeks of age for two weeks. Islets were pooled from 3 mice per genotype, 5 replicates per group. Values were normalized to insulin content in each sample, and are shown relative to secretion at high glucose by control mice. Error bars indicate S.D.

FIGS. 7A-D. p16 induction in the Insulin-rtTA/tet-p16 model. (A) Histogram of FACS analysis of p16 staining in Insulin⁺ islet cells from wt mice (black line), tetracycline-treated Insulin-rtTA/tet-p16 mice (red line) at 5 weeks of age, and 10-day old, Insulin-rtTA/tet-p16 not treated with tetracycline (blue line) mice. Low level tetracycline-independent expression is observed in 10d-old mice. (B) Histogram of stain of p16 in insulin-positive islet cells in wt (black line) mice, tetracycline-treated Insulin-rtTA/tet-p16 (red line) and non-treated Insulin-rtTA/tet-p16 (blue line) mice at 6 weeks of age. (C) Representative images of pancreatic sections of control and p16-expressing Insulin-rtTA/tet-p16 mice stained for Insulin (brown). Images reveal reduction in insulin staining indicating overall reduced β-cell mass due to p16 expression, including early tetracycline-independent expression. Each image represents a merge of multiple high-magnification images. (D) Bar graph showing body weights of control and p16-expressing mice following 10 days of p16 induction.

FIGS. 8A-C. p16 induction in the Pdx1-tTA/tet-p16 model. (A) FACS analysis of p16 staining of Insulin⁺ islet cells in wt mice (black line), induced (tet removed) Pdx1-tTA/tet-p16 mice (red line) and non-induced (tet maintained) Pdx1-tTA/tet-p16 (blue line) mice. (B) Images of pancreatic sections stained for nsulin (brown) of Pdx1-tTA and Pdx1-tTA/tet-p16 mice induced for 2 weeks, 2 months or 5 months. Stains reveal reduced β-cell mass at 5 months. Each image represents a merge of multiple high-magnification images. (C) Bar graphs of body weights of wt, Pdx1-tTA and Pdx1-tTA/tet-p16 mice, induced for 2 weeks.

FIGS. 9A-L. Increased glucose uptake and mitochondrial activity in p16-induced β-cells. (A) Bar graphs showing relative mRNA levels of the gck and aldob genes in β-cells expressing GFP (Insulin-rtTA/tet-GFP, white, n=2) or p16 together with GFP (Insulin-rtTA/tet-GFP/tet-p16, grey, n=3), following induction and sorting. Error bars indicate S.E.M. (B) Bar graphs showing glucose uptake in dissociated islet cells from control and p16-expressing mice, measured by FACS following incubation with the fluorescent glucose analog 2-NBDG for 30 minutes. Shown are mean fluorescence values of 5 independent islet samples, from p16-expressing and control islets, each pooled from 3 individual mice. Error bars indicate S.E.M. (C) Bar graphs showing relative mRNA levels of Ppargc1a, the gene encoding Pgc1α in GFP and p16-expressing sorted β-cells, as in FIG. 9A. (D) Images of western blots of Pgc1α in control and p16-expressing islets with Hsp90 shown as loading control. (E) Histogram of mitochondrial content in dissociated control and p16-expressing islet cells, as measured by FACS following MitoTracker staining. (F) Representative electron microscopy images of β-cells from control and p16-expressing islets. Mitochondria are indicated by arrows. (G) Bar graphs of mean mitochondrial area in p16-expressing β-cells and control β-cells, calculated as percentage per field based of electron microscopy images. >20 cells from 5 mice were scored in each group. Error bars indicate S.E.M. (H) Histogram of levels of the mitochondrial marker Atp5a in Insulin⁺ control and p16-expressing islet cells assessed by FACS. Red line shows p16⁺ cells only. (I) Bar graphs showing quantification of mitochondrial DNA content in equal numbers of GFP (Insulin-ftTA/tet-GFP, white) and p16 (Insulin-rtTA/tet-GFP/tet-p16, grey) sorted β-cells, isolated from >5 mice and pooled. Graph shows qPCR of the cytochrome b gene. Values were normalized to L1 repeat sequence levels as genomic DNA control. Values indicate mean of triplicate reactions ±S.E.M. (J) Histograms of mitochondrial membrane potential (ΔΨm) in dissociated GFP (Insulin-rtTA/tet-GFP) and p16 (Insulin-rtTA/tet-GFP/tet-p16) expressing β-cells measured by TMRE staining following incubation in low glucose (3 mM, left panel) and high glucose (20 mM, right panel). Cells were pooled from 5 mice in each group. The experiment was repeated three times. (K) Representative images of p16-expressing and control cells stained by TMRE at high glucose. (L) Line graph depicting oxygen consumption rates of control and p16-expressing islets. Dotted lines indicate times in which glucose (20 mM), FCCP, and rotenone+antimycin A were added. Values were normalized to islet insulin content in each sample, and are presented relative to basal levels of control islets. Shown are mean±S.E.M values of 5 replicates containing 40 islets each, pooled from 5 mice per group. The experiment was repeated 3 times. In all panels: *—P<0.05, **—P<0.005, ***—P<0.0005

FIGS. 10A-G. p16 drives elevated insulin secretion in mature mice. (A) Scatter plot showing GSIS of pancreatic islets of juvenile, 1 month-old mice, mature 6 months and 11 months-old mice, and geriatric, 27 month-old mice. Dots indicate individual mice, 3 replicates per mouse. Values were normalized to insulin content and are presented relative to mean secretion levels of 1 month-old mice at high glucose, defined as 1. Error bars indicate S.D. The experiment was repeated 3 times. (B) Histogram of FACS analysis of endogenous p16 expression in β-cells (Insulin⁺) from mice of indicated ages. (C) FSC histogram of β-cells (Insulin⁺) from 1 month and 6 month-old mice. Cells were pooled from 3 mice in each group. The experiment was repeated 5 times. (D) Histogram of SA-βGal stain of islet cells from 1 and 9 month-old mice. (E) Scatter plot showing GSIS of islets of 6 month-old wild type FVB mice and age-matched p16-null FVB mice. Graph includes mice assayed in 2 independent experiments. Values are presented relative to the secretion level of control islets at high-glucose, defined as 1. Error bars indicate S.D. (F) FSC-A histogram of β-cells (Insulin⁺) from mature control and p16 null mice. Cells were pooled from 3 mice in each group. (G) Scatter plot depicting GSIS of islets of 2 month-old MIP-CreER/CDK4^{+/lsl-R24C} and their sibling MIP-CreER/CDK4^{+/+} controls 2 weeks after Cre activation by tamoxifen treatment. Values are presented relative to the secretion level of control islets at high-glucose, defined as 1. Error bars indicate S.D. In all panels: *—P<0.05; ***—P<0.0005.

FIGS. 11A-C. Reduced β-cell proliferation during normal mouse maturation, and increased proliferation in mature p16-null mice. (A) Dot plot of FACS analysis of β-cells from 1 month-old mice co-stained for Ki67 and insulin. Shown are Insulin⁺ cells only. (B,C) Dot plots of FACS analysis of β-cells from (B) 6-month-old and (C) 12 month-old control FVB and Cdkn2a^{-/-} (p16KO) mice. Cells were pooled from 3 mice per group.

FIGS. 12A-B. Changes in GSIS during mouse aging—additional data. (A) Scatter plot of insulin secretion at low glucose (2.8 mM) by pancreatic islets of juvenile mice, 1 month-old and mature 6 month old, 11 month old and 27 month old, as shown in FIG. 10A with changed Y axis. Dots indicate individual mice, 3 replicates per mouse. Data was normalized to insulin content. (B) Bar graph showing fold increase in insulin secretion at high versus low glucose conditions, by pancreatic islets of 1 month-old, 6 month old, 11 month old and 27 month old wt mice, as well as Insulin-rtTA/tet-p16 (p16OE) and p16KO mice relative to their respective controls. Values were calculated based on data shown in FIGS. 5A, 10A, 10E.

FIG. 13. Lack of expression of human growth hormone in MIP-CreER mice. Micrographs of sections of MIP-CreER and control wild type mice were stained for growth hormone, to exclude protein expression from sequences contained in the transgene. A human pituitary section was used as a positive control.

FIGS. 14A-L. β-cell senescence in human islets. (A) Representative images of islet sections of a 5-month old (left panel) and a 45-year old (right panel) human subject stained for Insulin (blue) and p16 (green). (B) Bar graphs showing mean intensity of p16 expression in islets of juvenile (5 months to 10 years old, n=6) and adult (30 to 60 years old, n=5) human subjects, calculated by image analysis. >10 islets per subject were scored. Arbitrary units (a.u.) represent intensity per area normalized to staining levels in surrounding acinar cells. ***—P<0.0005 (C) Dot graph of relative p16 mRNA levels in islets of human subjects aged 6 months to 60 years (n=18) (D) Dot plot of FACS analysis of dissociated live human islets of a 42-year old subject, stained for insulin and p16. A p16-expressing fraction of β-cells (p16⁺/Insulin⁺) is observed. (E) FSC histograms of p16⁺ and p16-negative β-cells within the islet cell population of a 56-year old subject. Shown are Insulin⁺ cells. (F) Representative images of islet sections of a 5-month old (left panel) and a 51-year old (right panel) human subject stained for Insulin (blue) and pS6 (green). (G) Table showing the percentages of SA-βGal⁺ cells detected in live dissociated human islets. (H) Histogram of SA-βGal stain of live islet cells from representative adult human subject. (I) Histogram of SA-βGal stain of live islet cells from a juvenile human subject. (J) Histogram of FACS analysis of TMRE stained SA-βGal⁺ and SA-βGal⁻ dissociated human islet cells. The experiment was repeated three times. (K) Representative images of islet sections of a 5-year old (left panel) and a 45-year old (right panel) human subject stained for Insulin (blue) and the mitochondrial marker, Cox17 (green). (L) Bar graphs of mean intensity of Cox17 expression in juvenile (5 months to 10 years old, n=6) and adult (30 to 60 years old, n=5) human islets, normalized to Cox17 expression in surrounding acinar cells, calculated by image analysis. >10 islets per subject were scored. *—P<0.05.

FIGS. 15A-B. Increased expression of mitochondrial markers and phospho-S6 in human islets with age. (A) Representative images of islet sections of juvenile and adult human subjects at indicated ages stained for mitochondrial proteins. Increased staining levels are observed with age. Images present 3 islets from each subject. (B) Representative images of islet sections of juvenile and adult human subjects at the indicated ages stained for pS6.

FIGS. 16A-L. p16-induced senescence of human EndoC-βH2 cells leads to enhanced GSIS and is mediated by mTOR and PPARγ. (A) Histogram of FACS analysis of Ki67-stained EndoC-βH2 cells 3 weeks after infection with a lentivirus expressing GFP or both Cre and GFP; graphs display only GFP⁺ cells. Expression of Cre excises the large-T-antigen and hTert genes expressed in these cells, allowing reactivation of Rb and p53 and leading to cell cycle arrest. (B) Micrographs of SA-βGal activity (blue) in EndoC-βH2 cells expressing GFP or Cre+GFP, 3 weeks after infection. (C) Micrographs of EndoC-βH2 cells expressing GFP or Cre+GFP stained for insulin (red). (D) Bar graphs of insulin levels secreted by EndoC-βH2 cells expressing GFP (white), or Cre+GFP (grey) 3 weeks after infection, following incubation in low glucose (2.8 mM) and stimulation with high glucose (16.7 mM) for 1 hour. Secretion values were normalized to cell number, and are presented relative to insulin secretion levels of control cells at high glucose. Bars indicate mean of 4 replicates per group ±S.D. (E) FSC histograms of GFP-expressing and GFP+Cre-expressing cells indicating cell volumes. (F) Histogram showing FACS analysis of TMRE staining, indicating mitochondrial membrane potential, of same cells, incubated in high glucose (16.7 mM) for 1 hour. (G) Histogram of FACS analysis of 2-NBDG fluorescence, indicating glucose uptake, of cells infected with a Cre-expressing lentivirus or the pLKO empty virus control. (H) Micrographs of EndoC-βH2 cells that were first stably infected with pLKO-shp16, or with pLKO empty vector as control, and then infected with a Cre expressing lentivirus. Cells were stained for SA-βGal activity (blue) 3 weeks after infection. (I) Histogram of FACS analysis of TMRE staining of control and p16-silenced EndoC-βH2 cells 3 weeks after infection with a Cre-expressing lentivirus. (J) Bar graph of insulin levels secreted by control and p16-silenced EndoC-βH2 cells 3 weeks after infection with a Cre-expressing lentivirus, following incubation in low glucose (2.8 mM) and stimulation with high glucose (16.7 mM) for 1 hour. Secretion values were normalized to cell number, and are presented relative to insulin secretion levels of control cells at high glucose. Bars indicate mean of 5 replicates per group ±S.D. (K) Bar graph of insulin levels secreted by EndoC-βH2 cells infected with Cre and treated with the mTor kinase inhibitor Torin1 or with vehicle, for 3 weeks. Bars indicate mean of 5 replicates per group ±S.D. (L) Bar graph of insulin levels secreted by EndoC-βH2 cells infected with Cre and treated with the PPARγ inhibitor GW9662 or with vehicle, for 3 weeks. Bars indicate mean of 5 replicates per group ±S.D. In all panels: *—P<0.05; **—P<0.005; ***—P<0.0005.

FIGS. 17A-D. p16 expression levels in EndoC-βH2 cells. (A) Images of western blot analysis of p16 in EndoC-βH2 expressing GFP or Cre. (B) Images of western blot analysis of p16 levels in Cre expressing EndoC-βH2 (Cre), and in two samples of human islets (Human 1, 2) from middle aged subjects, indicating comparable levels. (C) Bar graph of mRNA levels of p16, the proliferation marker Ki67, and insulin, assessed by qRT-PCR in EndoC-βH2 cells expressing the pLKO vector (vector only), pLKO and Cre (vector+Cre) or shp16 and Cre. p16 levels go up in Cre expressing cells, but are reduced in shp16-expressing cells. Ki67 levels are reduced in Cre infected cells, and are not affected by p16 silencing. Insulin expression levels are reduced in p16-silenced cells. (D) Histogram of FACS analysis of Ki67 expression in the indicated cells, indicating that p16 silencing does not prevent cell-cycle arrest upon Cre infection.

FIGS. 18A-C. Cell size and mitochondrial activity in EndoC-βH2 cells following mTor or PPARγ inhibition. (A-B) Histograms of FACS analysis of FSC (A) and TMRE staining (B) of cells three weeks after infection with a control GFP vector or with Cre, and treatment with the mTor inhibitor Torin1 or with vehicle (v). (C) Histogram of FACS analysis of TMRE stained cells three weeks after infection with an empty pLKO vector or with Cre, and treatment with the PPARγ inhibitor GW9662, or with vehicle (v).

FIG. 19. Diagram summarizing the effects of p16-induced senescence on β cell function. Diagram of components of the senescence program contributing to increased insulin secretion are highlighted in red in the senescent β-cell (right panel) compared to juvenile β-cell (left panel).

### DETAILED DESCRIPTION OF THE INVENTION

The present invention provides modified insulin-secreting cell populations and compositions comprising same, methods and kits for increasing glucose induced insulin secretion and for treating a metabolic disease or disorder including, but not limited to, diabetes.

By one aspect, the present invention concerns a method for enhancing insulin secretion in response to glucose in an insulin secreting cell, the method comprising: subjecting an insulin producing cell to a condition that induces or increases expression of a senescence marker thereby enhancing insulin secretion in an insulin secreting cell.

Insulin secretion in response to glucose is a biological function of insulin secreting cells that is well known to individuals skilled in the art. Herein, the phenomenon is also referred to as glucose stimulated insulin secretion (GSIS). Glucose may be provided to the cell in vivo, through the blood stream, or in vitro by the addition of glucose to culture media. In the in vivo context, glucose may be ingested, or delivered directly to the blood stream by injection. An insulin secreting cells ability to respond to glucose is essential for blood sugar regulation and for creating glucose homeostasis in an organism.

Insulin Secreting Cells

The term “insulin secreting cell” refers to any cell capable of producing and secreting insulin. In some embodiments, the insulin secreting cell is a beta cell (β-cell). β-cells are found in islets in the pancreases.

In some embodiments, the cell is manipulated or engineered to secrete insulin. Such a cell may be isolated from a human subject or from a mammalian model organism. Examples of such cells include, but are not limited to, cells isolated from the pancreas, beta cells, pluripotent stem cells (PSCs) (embryonic or induced pluripotent cells) differentiated into insulin secreting cells, other stem cells (mesenchymal stem cells, hematopoietic stem cells, etc.) transdifferentiated into insulin secreting cells, cells transfected, or virally infected, or in any way made to expresses exogenous genes or proteins that result in insulin secretion.

The term “pluripotent stem cell” refers to cells capable of differentiating, or being differentiated by means known to one ordinary in the art, into cells of any lineage. The term “embryonic stem cell” refers to stem cells derived from the undifferentiated inner mass of an embryo. Such cells are pluripotent, and capable of differentiating, or being differentiated by means known to one ordinary in the art, into cells of any lineage. In order for an ESC to be considered undifferentiated, it must continue to express stem cell markers or not express markers of differentiated cells.

The term PSC refers to pluripotent stem cells regardless of their derivation, the term PSC encompasses the terms ESC and iPSC, as well as the term embryonic germ stem cells (EGSC), which are another example of a PSC. PSCs may be in the form of an established cell line, they may be obtained directly from primary embryonic tissue, or they may be derived from a somatic cell. PSCs can be target cells of the methods described herein.

Human ESC lines are listed in the NIH Human Embryonic Stem Cell Registry, e.g. hESBGN-01, hESBGN-02, hESBGN-03, hESBGN-04 (BresaGen, Inc.); HES-1, HES-2, HES-3, HES-4, HES-5, HES-6 (ES Cell International); Miz-hES1 (MizMedi Hospital-Seoul National University); HSF-1, HSF-6 (University of California at San Francisco); and HI, H7, H9, HI 3, H14 (Wisconsin Alumni Research Foundation (WiCell Research Institute)). Stem cells of interest also include embryonic stem cells from other primates, such as Rhesus stem cells and marmoset stem cells. The stem cells may be obtained from any mammalian species, e.g. human, equine, bovine, porcine, canine, feline, rodent, e.g. mice, rats, hamster, primate, etc. (Thomson et al. (1998) Science 282:1145; Thomson et al. (1995) Proc. Natl. Acad. Sci USA 92:7844; Thomson et al. (1996) Biol. Reprod. 55:254; Shamblott et al., Proc. Natl. Acad. Sci. USA 95:13726, 1998). In culture, ESCs typically grow as flat colonies with large nucleo-cytoplasmic ratios, defined borders and prominent nucleoli. In addition, ESCs express SSEA-3, SSEA-4, TRA-1-60, TRA-1-81, and Alkaline Phosphatase, but not SSEA-1. Examples of methods of generating and characterizing ESCs may be found in, for example, U.S. Pat. No. 7,029,913, U.S. Pat. No. 5,843,780, and U.S. Pat. No. 6,200,806. Methods for proliferating hESCs in the undifferentiated form are described in WO 99/20741, WO 01/51616, and WO 03/020920.

By “embryonic germ stem cell” (EGSC) or “embryonic germ cell” or “EG cell” is meant a PSC that is derived from germ cells and/or germ cell progenitors, e.g. primordial germ cells, i.e. those that would become sperm and eggs. Embryonic germ cells (EG cells) are thought to have properties similar to embryonic stem cells as described above. Examples of methods of generating and characterizing EG cells may be found in, for example, U.S. Pat. No. 7,153,684; Matsui, Y., et al., (1992) Cell 70:841; Shamblott, M., et al. (2001) Proc. Natl. Acad. Sci. USA 98: 113; Shamblott, M., et al. (1998) Proc. Natl. Acad. Sci. USA, 95:13726; and Koshimizu, U., et al. (1996) Development, 122:1235.

By “induced pluripotent stem cell” or “iPSC” it is meant a PSC that is derived from a cell that is not a PSC (i.e., from a cell this is differentiated relative to a PSC). iPSCs can be derived from multiple different cell types, including terminally differentiated cells. iPSCs have an ES cell-like morphology, growing as flat colonies with large nucleo-cytoplasmic ratios, defined borders and prominent nuclei. In addition, iPSCs express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26al, TERT, and zfp42. Examples of methods of generating and characterizing iPSCs may be found in, for example, U.S. Patent Publication Nos. US20090047263, US20090068742, US20090191159, US20090227032, US20090246875, and US20090304646. Generally, to generate iPSCs, somatic cells are provided with reprogramming factors (e.g. Oct4, SOX2, KLF4, MYC, Nanog, Lin28, etc.) known in the art to reprogram the somatic cells to become pluripotent stem cells.

In some embodiments, the cell is a cell of an immortalized β-cell line. Such cells lines, and the media necessary for their culture is well known in the art. Some non-limiting examples of such lines, include: MIN6, INS-1E, PANC1, Blox5, TRM-1, CM, CCL-104, CRL-2989, Beta-TC-3 or 6 (American Type Culture Collection), EndoC-BH1, EndoC-BH2, EndoC-BH3 (Endocells).

In some embodiments, the β-cell is grown in culture in β-cell growth media. Such media is well known in the art and can be purchased from purveyors of culture media. Non-limiting examples of (3-cell growth media include: RPMI 1640, DMEM or CMRL 1066. In some embodiments, β-cell growth media is supplemented in order to support β-cell growth. Non-limiting examples of such supplements include: human growth hormone, BSA, beta-2-mercaptoethanol, niotinamide, transferrin, selenite, fetal calf serum, GLP or glucose.

In some embodiments, the β-cells are cultured on coated plates. Examples of such coating include but are not limited to gelatin, laminin, polylysine, fibronectin and Matrigel.

In some embodiments, the cell is engineered to secrete insulin by exogenous expression of one or more genes necessary for insulin secretion. Examples of such genes include, but are not limited to, INS, HNF1, PDX1, MAFA, NEUROD1, HNF4α, TCF3, HNF1β, HNF3β, and GCK.

In some embodiments, the cell is first fasted (deprived of glucose for a period of time) before glucose is provided and insulin secretion is induced. In some embodiments, the cell is maintained in conditions of low glucose before high levels of glucose are provided to the cell.

In some embodiments, low glucose is any one of the following ranges: 0.5-6.0 mM glucose, 1.0-6.0 mM glucose, 1.5-6.0 mM glucose, 2.0-6.0 mM glucose, 2.5-6.0 mM glucose, 3.0-6.0 mM glucose, 3.5-6.0 mM glucose, 4.0-6.0 mM glucose, 4.5-6.0 mM glucose, 5.0-6.0 mM glucose, 5.5-6.0 mM glucose, 0.5-5.5 mM glucose, 1.0-5.5 mM glucose, 1.5-5.5 mM glucose, 2.0-5.5 mM glucose, 2.5-5.5 mM glucose, 3.0-5.5 mM glucose, 3.5-5.5 mM glucose, 4.0-5.5 mM glucose, 4.5-5.5 mM glucose, 5.0-5.5 mM glucose, 0.5-5.0 mM glucose, 1.0-5.0 mM glucose, 1.5-5.0 mM glucose, 2.0-5.0 mM glucose, 2.5-5.0 mM glucose, 3.0-5.0 mM glucose, 3.5-5.0 mM glucose, 4.0-5.0 mM glucose, 4.5-5.0 mM glucose, 0.5-4.5 mM glucose, 1.0-4.5 mM glucose, 1.5-4.5 mM glucose, 2.0-4.5 mM glucose, 2.5-4.5 mM glucose, 3.0-4.5 mM glucose, 3.5-4.5 mM glucose, 4.0-4.5 mM glucose, 0.5-4.0 mM glucose, 1.0-4.0 mM glucose, 1.5-4.0 mM glucose, 2.0-4.0 mM glucose, 2.5-4.0 mM glucose, 3.0-4.0 mM glucose, 3.5-4.0 mM glucose, 0.5-3.5 mM glucose, 1.0-3.5 mM glucose, 1.5-3.5 mM glucose, 2.0-3.5 mM glucose, 2.5-3.5 mM glucose, 3.0-3.5 mM glucose, 0.5-3.0 mM glucose, 1.0-3.0 mM glucose, 1.5-3.0 mM glucose, 2.0-3.0 mM glucose, 2.5-3.0 mM glucose, 0.5-2.5 mM glucose, 1.0-2.5 mM glucose, 1.5-2.5 mM glucose, 2.0-2.5 mM glucose, 0.5-2.0 mM glucose, 1.0-2.0 mM glucose, 1.5-2.0 mM glucose, 0.5-1.5 mM glucose, 1.0-1.5 mM glucose, 0.5-1.0 mM glucose. Each possibility represents a separate embodiment of the present invention

In some embodiments, low glucose is less than 3.0 mM glucose, less than 3.5 mM glucose, less than 4.0 mM glucose, less than 4.5 mM glucose, less than 5.0 mM glucose, less than 5.5 mM glucose, or less than 6.0 mM glucose. Each possibility represents a separate embodiment of the present invention.

In some embodiments, high glucose is any one of the following ranges: 9.0-20.0 mM glucose, 10.0-20.0 mM glucose, 11.0-20.0 mM glucose, 12.0-20.0 mM glucose, 13.0-20.0 mM glucose, 14.0-20.0 mM glucose, 15.0-20.0 mM glucose, 16.0-20.0 mM glucose, 17.0-20.0 mM glucose, 18.0-20.0 mM glucose, 19.0-20.0 mM glucose, 9.0-19.0 mM glucose, 10.0-19.0 mM glucose, 11.0-19.0 mM glucose, 12.0-19.0 mM glucose, 13.0-19.0 mM glucose, 14.0-19.0 mM glucose, 15.0-19.0 mM glucose, 16.0-19.0 mM glucose, 17.0-19.0 mM glucose, 18.0-19.0 mM glucose, 9.0-18.0 mM glucose, 10.0-18.0 mM glucose, 11.0-18.0 mM glucose, 12.0-18.0 mM glucose, 13.0-18.0 mM glucose, 14.0-18.0 mM glucose, 15.0-18.0 mM glucose, 16.0-18.0 mM glucose, 17.0-18.0 mM glucose, 9.0-17.0 mM glucose, 10.0-17.0 mM glucose, 11.0-17.0 mM glucose, 12.0-17.0 mM glucose, 13.0-17.0 mM glucose, 14.0-17.0 mM glucose, 15.0-17.0 mM glucose, 16.0-17.0 mM glucose, 9.0-16.0 mM glucose, 10.0-16.0 mM glucose, 11.0-16.0 mM glucose, 12.0-16.0 mM glucose, 13.0-16.0 mM glucose, 14.0-16.0 mM glucose, 15.0-16.0 mM glucose, 9.0-15.0 mM glucose, 10.0-15.0 mM glucose, 11.0-15.0 mM glucose, 12.0-15.0 mM glucose, 13.0-15.0 mM glucose, 14.0-15.0 mM glucose, 9.0-14.0 mM glucose, 10.0-14.0 mM glucose, 11.0-14.0 mM glucose, 12.0-14.0 mM glucose, 13.0-14.0 mM glucose, 9.0-13.0 mM glucose, 10.0-13.0 mM glucose, 11.0-13.0 mM glucose, 12.0-13.0 mM glucose, 9.0-12.0 mM glucose, 10.0-12.0 mM glucose, 11.0-12.0 mM glucose, 9.0-11.0 mM glucose, 10.0-11.0 mM glucose, 9.0-10.0 mM glucose. Each possibility represents a separate embodiment of the present invention.

In some embodiments, high glucose is at least 9.0, at least 9.5, at least 10.0, at least 10.5, at least 11.0, at least 11.5, at least 12.0, at least 12.5, at least 13.0, at least 13.5, at least 14.0, at least 14.5, at least 15.0, at least 15.5, at least 16.0, at least 16.5, at least 17.0, at least 17.5, at least 18.0, at least 18.5, at least 19.0, at least 19.5 or at least 20.0 mM glucose. Each possibility represents a separate embodiment of the present invention.

Senescence

The terms “senescence” and “cellular senescence” are synonymous and interchangeable, and as used herein refer a cell that no longer replicates, but remains metabolically active. Such a cell will adopt some (but not necessarily all) of several prominent features, including: increased cell size, increased secretion of extracellular matrix factors and cytokines (known as the senescence-associated secretory phenotype, SASP), increased protein production and degradation through lysosomal activity, and modified carbohydrate metabolism involving increased glucose uptake and mitochondrial respiration. Senescence may be detected through several markers, which, herein, are referred to as “senescence markers” or “senescence-associated markers”.

In some embodiments, the senescence-associated markers is senescence-associated β-galactosidase (SA-beta-Gal) activity, which detects increased lysosomal activity. In additional embodiments, senescence may be detected via determining expression of different central molecular inducers of senescence either individually or in combination. Examples of such senescence-associated markers include but are not limited to: p16^{Ink4a}, p14^{ARF} (p19^{ARF} in mouse), p53, p21, and dephosphorylated Rb. Additional markers, including PAI1 (SerpinE2), Dec1, Lamp2A and HP1gamma. In some embodiments, the senescence-associated markers are DNA damage markers including gammaH2AX and 53BP. In some settings senescent cells display a nucleus characterized by senescence-associated heterochromatin foci (SAHF) or DNA segments with chromatin alterations reinforcing senescence (DNA-SCARS).

In some embodiments, the senescence-associated marker is the absence or loss of expression of a marker of cellular division. In some embodiments, the marker of cellular division is selected from the group consisting of: Ki67, BrdU incorporation, CDC47 and phosphorylated Histone H3.

In some embodiments, the senescence-associated marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

In some embodiments, the condition that induces or increases expression of a senescence marker is expressing within the cell one or more genes selected from p16-Ink4a, p21, p14ARF, p53 and Rb, irradiating the cell, inducing oxidative stress in the cell, and inducing DNA damage in the cell. In some embodiments, the DNA damage is induced through drug treatment. In some embodiments, the condition that induces or increases expression of a senescence marker is expressing within the cell p16-Ink4a. In some embodiments, the conditions are applied at levels and for a time period sufficient to cause senescence without causing cellular death. In some embodiments, the conditions are applied at levels and for a time period sufficient to cause senescence in parallel to cellular death. In some embodiments, the irradiation is electromagnetic irradiation. In some embodiments, inducing DNA damage comprises exposing the cell to DNA damaging agents including radiomimetics, drugs causing DNA damage such as Doxorubicin or drugs/agents disrupting chromatin structure such as 5-azacytidine or histone deacetylases for example. In some embodiments, inducing oxidative stress in the cell comprises exposing the cell to oxidizing agents such as hydrogen peroxide, nitric oxide or menadione. In some embodiments, inducing oxidative stress comprises depriving the cell of oxygen, damaging the cells mitochondria, or any other treatment that disrupts oxidative phosphorylation in the cell and/or produces free radicals.

In some embodiments, the insulin secreting cell is present in a living organism such as a human subject, and in such a case the conditions that induce or increase senescence may be applied to the full body of the individual or selectively or preferentially applied to the pancreas in general or to insulin secreting cells in particular (for example by directing irradiation to the region of the pancreas, or by targeted delivery to the pancreas or to beta cells in particular). This application to the full body of the individual or to cells of an individual may be carried out in order to treat any clinical condition wherein glucose stimulated insulin secretion is to be enhanced. In some embodiments, these conditions are diabetes, as well as other metabolic disorders.

The terms, “identity” and “cellular identity” are synonymous and interchangeable, and as used herein refer to a cell's phenotypic differentiated state. As it pertains to the cells of the invention, their cellular identity is that of insulin secreting cells. In some embodiments, retention of cellular identity comprises retaining the ability to secrete insulin in response to glucose. In some embodiments, retention of cellular identity comprises retention of expression of a marker of cellular identity. In some embodiments, the marker of cellular identity is a marker of β-cell identity. In some embodiments, the marker is selected from the group consisting of: Nkx6.1, Pdx1, Chga, Ins, Mafa, Math, Nkx2.2, Neurod1, Pax6, Neurog3, Slc2a2, Kcnj11, Abcc8, Ffar1, and Pcsk1.

In some embodiments, confirmation of a retention of cellular identity is performed. In some embodiments, confirmation comprises measuring a marker of cellular identity. In some embodiments, the marker is selected from the group consisting of: Nkx6.1, Pdx1, Chga, Ins, Mafa, Math, Nkx2.2, Neurod1, Pax6, Neurog3, Slc2a2, Kcnj11, Abcc8, Ffar1, and Pcsk1. In some embodiments, the marker is a mRNA marker, and measuring is measuring mRNA levels by methods such as are common in the art (i.e. PCR, in situ hybridization, etc.). In some embodiments, the marker is a protein marker, and measuring is measuring protein levels in the cell or secreted by the cell by methods such as are common in the art (i.e. immunoblot, immunostaining, ELISA, etc.).

In some embodiments, an increase of insulin secretion in response to glucose from the cell of the invention as compared to a control cell is confirmed. In some embodiments, the control cell is the same insulin secreting cell without subjecting the cell to conditions that induce or increase expression of a senescence marker.

As a non-limiting example, the increase of insulin secretion in response to glucose from the cell of the invention, as compared to a control cell, is at least doubling of insulin secretion. In some embodiments, the increase is at least 5%, is at least 10%, at least 20%, at least 30%, at least 40%, at least 50%, at least 60%, at least 70%, at least 80%, at least 90%, at least 100%, at least 125%, at least 150%, at least 175%, at least 200%, at least 300%, at least 400%, or at least 500% increase in insulin secretion in response to glucose. Each possibility represents a separate embodiment of the present invention.

In some embodiments, the cells to be administered to the subject are allogenic to the subject. In some embodiments, the cells to be administered to the subject are autogenic to the subject. In some embodiments, the cells to be administered to the subject are autologous to the subject.

In some embodiments, the subjecting the cell to a condition that induces or increases expression of a senescence marker and does not induce DNA damage is expressing p16-Ink4a within the cell. In some embodiments, this expressing is expressing mammalian p16-Ink4a mRNA. In some embodiments, this expressing is expressing human p16-Ink4a mRNA. In some embodiments, this expressing is expressing human p16-Ink4a cDNA. In some embodiments, human p16-Ink4a has the following nucleic acid sequence:

(SEQ ID NO: 1)
ATGGAGCCGGCGGCGGGGAGCAGCATGGAGCCTTCGGCTGACTGGCTGGC
CACGGCCGCGGCCCGGGGTCGGGTAGAGGAGGTGCGGGCGCTGCTGGAGG
CGGGGGCGCTGCCCAACGCACCGAATAGTTACGGTCGGAGGCCGATCCAG
GTCATGATGATGGGCAGCGCCCGAGTGGCGGAGCTGCTGCTGCTCCACGG
CGCGGAGCCCAACTGCGCCGACCCCGCCACTCTCACCCGACCCGTGCACG
ACGCTGCCCGGGAGGGCTTCCTGGACACGCTGGTGGTGCTGCACCGGGCC
GGGGCGCGGCTGGACGTGCGCGATGCCTGGGGCCGTCTGCCCGTGGACCT
GGCTGAGGAGCTGGGCCATCGCGATGTCGCACGGTACCTGCGCGCGGCTG
CGGGGGGCACCAGAGGCAGTAACCATGCCCGCATAGATGCCGCGGAAGGT
CCCTCAGACATCCCCGATTGA.

In some embodiments, this expressing is expressing mouse p16-Ink4a mRNA. In some embodiments, this expressing is expressing mouse p16-Ink4a cDNA. In some embodiments, mouse p16-Ink4a has the following nucleic acid sequence:

(SEQ ID NO: 2)
ATGGAGTCCGCTGCAGACAGACTGGCCAGGGCGGCGGCCCAGGGCCGTGT
GCATGACGTGCGGGCACTGCTGGAAGCCGGGGTTTCGCCCAACGCCCCGA
ACTCTTTCGGTCGTACCCCGATTCAGGTGATGATGATGGGCAACGTTCAC
GTAGCAGCTCTTCTGCTCAACTACGGTGCAGATTCGAACTGCGAGGACCC
CACTACCTTCTCCCGCCCGGTGCACGACGCAGCGCGGGAAGGCTTCCTGG
ACACGCTGGTGGTGCTGCACGGGTCAGGGGCTCGGCTGGATGTGCGCGAT
GCCTGGGGTCGCCTGCCGCTCGACTTGGCCCAAGAGCGGGGACATCAAGA
CATCGTGCGATATTTGCGTTCCGCTGGGTGCTCTTTGTGTTCCGCTGGGT
GGTCTTTGTGTACCGCTGGGAACGTCGCCCAGACCGACGGGCATAGCTTC
AGCTCAAGCACGCCCAGGGCCCTGGAACTTCGCGGCCAATCCCAAGAGCA
GAGCTAA

In some embodiments, this expressing is expressing mammalian p16-Ink4a protein. In some embodiments, this expressing is expressing human p16-Ink4a protein. In some embodiments, human p16-Ink4a has the following protein sequence:

(SEQ ID NO: 3)
MEPAAGSSMEPSADWLATAAARGRVEEVRALLEAGALPNAPNSYGRRPIQ
VMMMGSARVAELLLLHGAEPNCADPATLTRPVHDAAREGFLDTLVVLHRA
GARLDVRDAWGRLPVDLAEELGHRDVARYLRAAAGGTRGSNHARIDAAEG
PSDIPD.

In some embodiments, this expressing is expressing mouse p16-Ink4a protein. In some embodiments, mouse p16-Ink4a has the following protein sequence:

(SEQ ID NO: 4)
MESAADRLARAAAQGRVHDVRALLEAGVSPNAPNSFGRTPIQVMMMGNVH
VAALLLNYGADSNCEDPTTFSRPVHDAAREGFLDTLVVLHGSGARLDVRD
AWGRLPLDLAQERGHQDIVRYLRSAGCSLCSAGWSLCTAGNVAQTDGHSF
SSSTPRALELRGQSQEQS.

Expressing of a gene within a cell is well known to one skilled in the art. It can be carried out by, among many methods, transfection, viral infection, or direct alteration of the cell's genome. In some embodiments, the gene is in an expression vector such as plasmid or viral vector. One such example of an expression vector containing p16-Ink4a is the mammalian expression vector pCMV p16 INK4A available from Addgene.

A vector nucleic acid sequence generally contains at least an origin of replication for propagation in a cell and optionally additional elements, such as a heterologous polynucleotide sequence, expression control element (e.g., a promoter, enhancer), selectable marker (e.g., antibiotic resistance), poly-Adenine sequence.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter.

In some embodiments, the gene is operably linked to a promoter. The term “operably linked” is intended to mean that the nucleotide sequence of interest is linked to the regulatory element or elements in a manner that allows for expression of the nucleotide sequence (e.g. in an in vitro transcription/translation system or in a host cell when the vector is introduced into the host cell).

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like.

The term “promoter” as used herein refers to a group of transcriptional control modules that are clustered around the initiation site for an RNA polymerase i.e., RNA polymerase II. Promoters are composed of discrete functional modules, each consisting of approximately 7-20 bp of DNA, and containing one or more recognition sites for transcriptional activator or repressor proteins. In some embodiments, the gene is operably linked to a pancreas-specific promoter. In some embodiments, the gene is operably linked to a beta cell-specific promoter. Examples of such promoters include, but are not limited to, mammal PDX1, GCK, SLC2A2, PPY, SST, FOXA2, PFT1A, SOX9 promoter, mouse Ins1, or Ins2 promoters and human INS promoter. In some embodiments, the endogenous promoter has been modified to increase, decrease or otherwise modify expression. In some embodiments, the gene is operably linked to a beta cell-specific insulin-inducible promoter. An example of a plasmid containing such a promoter includes but is not limited to, the pGL3.hINS-363 3× plasmid.

In some embodiments, nucleic acid sequences are transcribed by RNA polymerase II (RNAP II and Pol II). RNAP II is an enzyme found in eukaryotic cells. It catalyzes the transcription of DNA to synthesize precursors of mRNA and most snRNA and microRNA.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available, e.g., from Invitrogen, pCI which is available, e.g., from Promega, pMbac, pPbac, pBK-RSV and pBK-CMV which are available, e.g., from Strategene, pTRES which is available, e.g., from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p205. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

In one embodiment, plant expression vectors are used. In one embodiment, the expression of a polypeptide coding sequence is driven by a number of promoters. In some embodiments, viral promoters such as the 35S RNA and 19S RNA promoters of CaMV [Brisson et al., Nature 310:511-514 (1984)], or the coat protein promoter to TMV [Takamatsu et al., EMBO J. 6:307-311 (1987)] are used. In another embodiment, plant promoters are used such as, for example, the small subunit of RUBISCO [Coruzzi et al., EMBO J. 3:1671-1680 (1984); and Brogli et al., Science 224:838-843 (1984)] or heat shock promoters, e.g., soybean hsp17.5-E or hsp17.3-B [Gurley et al., Mol. Cell. Biol. 6:559-565 (1986)]. In one embodiment, constructs are introduced into plant cells using Ti plasmid, Ri plasmid, plant viral vectors, direct DNA transformation, microinjection, electroporation and other techniques well known to the skilled artisan. See, for example, Weissbach & Weissbach [Methods for Plant Molecular Biology, Academic Press, NY, Section VIII, pp 421-463 (1988)]. Other expression systems such as insects and mammalian host cell systems, which are well known in the art, can also be used by the present invention.

It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

A person with skill in the art will appreciate that a gene can also be expressed from a nucleic acid construct administered to the individual employing any suitable mode of administration, described hereinabove (i.e., in vivo gene therapy). In one embodiment, the nucleic acid construct is introduced into a suitable cell via an appropriate gene delivery vehicle/method (transfection, transduction, homologous recombination, etc.) and an expression system as needed and then the modified cells are expanded in culture and returned to the individual (i.e., ex vivo gene therapy).

In some embodiments, transformed cells are cultured under effective conditions, which allow for the expression of high amounts of recombinant polypeptide. In some embodiments, effective culture conditions include, but are not limited to, effective media, bioreactor, temperature, pH and oxygen conditions that permit protein production. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

General methods in molecular and cellular biochemistry, such as may be useful for carrying out these techniques can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998).

By another aspect, there is provided a composition comprising a population of modified insulin secreting cells and a pharmaceutically acceptable carrier, said modified insulin secreting cells express one or more senescence-associated marker.

In some embodiments, the senescence marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A and HP1gamma.

In some embodiments, the modified insulin secreting cells secrete insulin in response to glucose at at-least 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0 times the level that unmodified insulin secreting cells do. Each possibility represents a separate embodiment of the present invention. In some embodiments, said modified cells secrete insulin in response to glucose at at-least twice the level that unmodified insulin secreting cells do. In some embodiments, the unmodified cell is the same insulin secreting cell which does not comprise expression of a senescence-associated marker.

In some embodiments, the subjecting does not induce DNA damage in the cell. The term “DNA damage” as used here in, refers to an alteration in the chemical structure of DNA, such as a break, a nick, a missing base, a chemical change to the DNA backbone or a mutation of one base to another. DNA damage does not refer to an intentional alteration of the genome, such as deleting or adding a gene or part of a gene such as is common in the art. In some embodiments, said subjecting without inducing DNA damage is expressing p16-Ink4a within the cell.

Therapeutic Use

By another aspect, there is provided a method of treating a metabolic syndrome (e.g., diabetes) in a subject in need thereof, the method comprising, providing an insulin producing cell, subjecting the cell to a condition that induces or increases expression of a senescence marker and does not induce DNA damage, thereby increasing glucose stimulated insulin secretion in the cell, and administering the cell to the subject, thereby treating a metabolic syndrome in the subject.

The phrase “administering” as used herein, refers to the therapeutic introduction of a cell or a pharmaceutical composition comprising same, to a subject. Administration may take place by any route that allows the cells to treat the metabolic syndrome (e.g., diabetes). In some embodiments, the cell or pharmaceutical composition comprising same, is directly administered to the pancreas of the patient through a variety of modes including, but not limited to, pancreatic injection, intravenous injection, and surgical administration.

In some embodiments, the subject suffers from diabetes type I, diabetes type II or another metabolic syndrome. The term “diabetes” refers to the metabolic disease diabetes mellitus. In some embodiments, this refers to type I diabetes, also known as insulin-dependent diabetes mellitus. In other embodiments, this refers to type II diabetes, also known as adult onset diabetes mellitus.

Other metabolic syndromes refer to any disease or disorder in which insulin, or glucose homeostasis plays a role. In some embodiments, diseases of glucose homeostasis include but are not limited to hypoglycemia, hyperglycemia.

In some embodiments, the disease of glucose homeostasis is Huntington's disease. Huntington's disease has been reported as having symptoms of abnormalities in insulin sensitivity and insulin secretion (Lalic et al., 2008, Archives of Neurology, 4:476-480; Nambron et al., 2016, PLoS One, 11(1):e0146480).

In some embodiments, the patient can be further treated with pharmaceutical agents or bioactives that facilitate the survival and function of the transplanted cells. These agents may include, for example, insulin, members of the TGF-β family, including TGF-βI, 2, and 3, bone morphogenic proteins (BMP-2, -3, -4, -5, -6, -7, -11, -12, and -13), fibroblast growth factors-1 and -2, platelet-derived growth factor-AA, and -BB, platelet rich plasma, insulin growth factor (ICE-I, II) growth differentiation factor (GDF-5, -6, -7, -8, -10, -15), vascular endothelial cell-derived growth factor (VEGF), pleiotrophin, endothelin, among others. Other pharmaceutical compounds can include, for example, nicotinamide, glucagon like peptide-I (GLP-1) and II, GLP-1 and 2, mimetibody, Exendin-4. retinoic acid, parathyroid hormone, or MAPK inhibitors.

As used herein, the term “carrier,” “excipient,” or “adjuvant” refers to any component of a pharmaceutical composition that is not the active agent. It can refer to any non-toxic, inert solid, semi-solid liquid filler, diluent, encapsulating material, formulation auxiliary of any type, or simply a sterile aqueous medium, such as saline.

Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations.

Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations.

Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety.

Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety.

The carrier may comprise, in total, from about 0.1% to about 99.99999% by weight of the pharmaceutical compositions presented herein.

In some embodiments, the insulin producing cell is subjected to conditions that induce or increase expression of a senescence marker ex vivo. In some embodiments, the subjecting occurs in vivo. In some embodiments, the cell is subjected to conditions that do not induce DNA damage so as to render the cells safe for transplant into a subject. In some embodiments, the condition does induce DNA damage, but the cells are further subjected to testing to determine that they are safe for transplantation into a subject. In some embodiments, safe for transplantation refers to the fact that the cells are not at an increased risk of developing into a tumor.

The terms “as in” and “as compared to” are synonymous and interchangeable, and refer to a comparison of a factor in two conditions. In some embodiments, this involves routine experimentation as is known to one skilled in the art, to measure the factor in both conditions. In some embodiments, this comparing entails measuring expression levels of a gene, nucleic acid, or protein in two cells and comparing which cell has higher or lower expression and determining the magnitude of the difference.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods

Mice.

To generate tetracycline-inducible p16^{Ink4a} (tet-p16) mice, Flp/FRT-mediated recombination was conducted in KH2 embryonic stem (ES) cells, as previously described (Tokarsky-Amiel et al., Cancer Research, 2013, 73:2829-39). tet-p16 and tet-GFP mice (mixed C57BL/6 and 129Sv background) were crossed with Insulin-rtTA (ICR background) or Pdx1-tTA mice (mixed ICR background). For transgene induction using the Insulin-rtTA driver (tet on) 500 mg/L tetracycline was added to the drinking water of double-transgenic mice and sibling control single-transgenics for the indicated durations. Littermate control mice carrying either the Insulin-rtTA transgene only, or the Insulin-rtTA/tet-GFP transgenes (as indicated in Figures) received identical drug treatments. For induction using the Pdx1-tTA (tet off) driver, tetracycline was removed at the experiment initiation. Littermate controls carrying no transgene, Pdx1-tTA and Pdx1-tTA/tet-p16 mice were kept on tetracycline concurrently from inception until the experiment initiation. p16^{Ink4a} null mice (FVB background) were obtained from the NCI Mouse Repository. CDK4^{+/lsl-R24C} mice were crossed with MIP-CreER mice (both on mixed ICR background). Two daily doses of 8 mg Tamoxifen (Sigma, 20 mg/ml in corn oil) were injected subcutaneously to 6-week old mice to obtain β-cell specific activation of CDK4^{R24C}. C57BL/6 and FVB mice were obtained from Harlan, Israel. Glucose tolerance, and in vivo GSIS were performed as previously described (Nir et al., Journal of Clinical Investigation, 2007, 117:2553-61): fasted mice received glucose by intraperitoneal injection (2 mg/kg), and blood glucose or serum insulin levels were measured at the indicated time-points. For insulin tolerance testing, fasted mice received 0.75 u/Kg insulin (Humalog) injected intraperitoneally, and blood glucose was measured at indicated time-points. All experiments were conducted using mouse litter batches, and where necessary experimental groups were comprised of multiple litters to allow statistical power. Two-sided Student's t-tests were used to compare mouse groups, under the assumption of normal distribution and observance of similar variance. Experiments using tet-p16 mice included both males and females; only males were used for aging and p16-null experiments for convenience. No statistical method was used to pre-determine sample size. The experiments were not randomized. There was no blinded allocation during experiments and outcome assessment. The joint ethics committee (IACUC) of the Hebrew University and Hadassah Medical Center approved the study protocol for animal welfare. The Hebrew University is an AAALAC International accredited institute.

Ex Vivo Glucose Stimulated Insulin Secretion (GSIS).

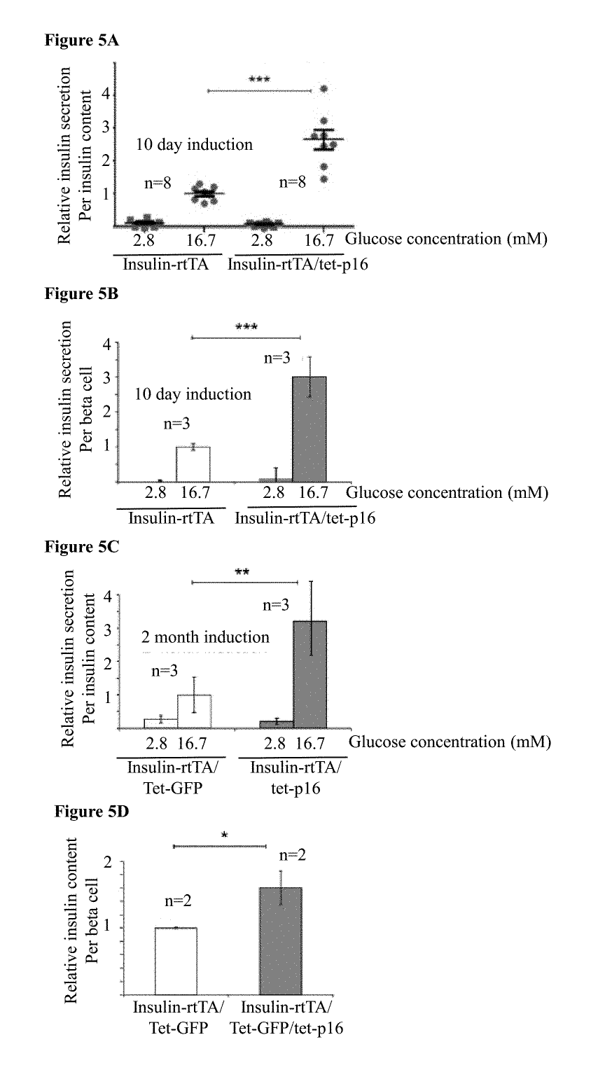

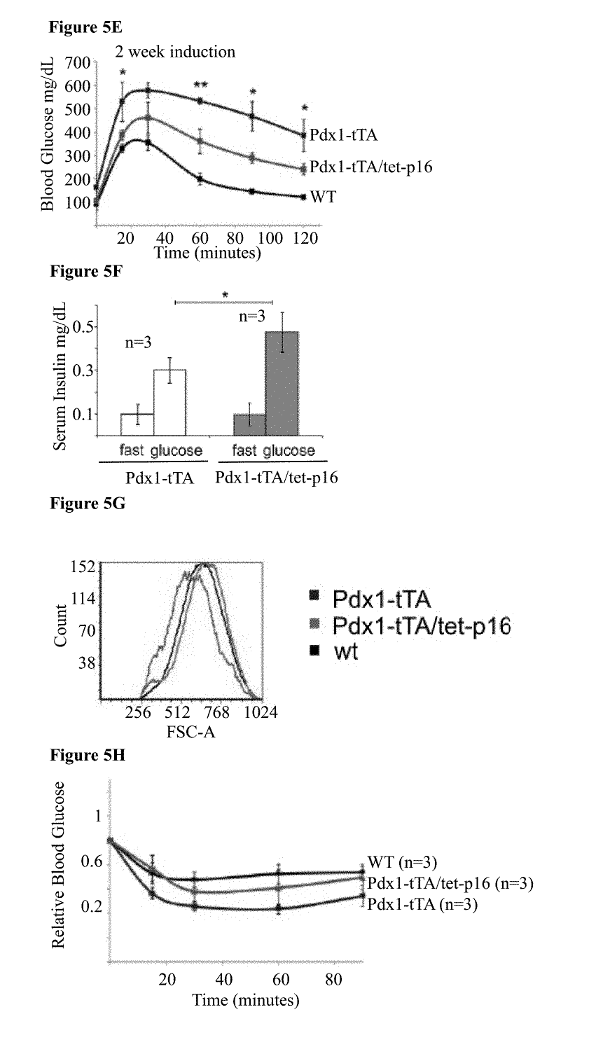

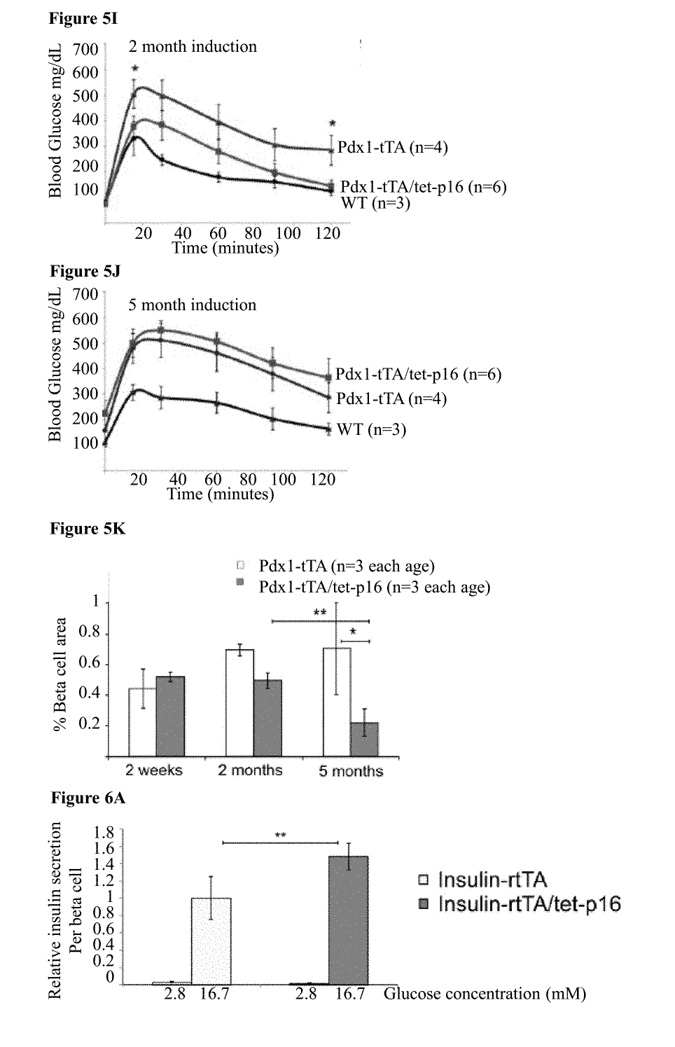

Islets were isolated from whole pancreata using Collagenase P (Roche) injected to the pancreatic duct followed by Histopaque gradient (Sigma). Islets were incubated overnight in RPMI-1640 medium supplemented with 10% fetal bovine serum, L-glutamine and penicillin/streptomycin in a 37° C. 5% CO₂ incubator. Islets were handpicked and placed in basal Krebs buffer containing 2.8 mM glucose, and then transferred into Krebs solution containing 16.7 mM glucose. After 1 hour incubation at 37° C., islets were pelleted and supernatants were collected. The pellet was solubilized to assess intracellular insulin content. Insulin levels were measured by ELISA (Crystal Chem Inc). Values were normalized to insulin levels in the subsequently lysed islets, indicative of β-cell number in each sample. Islets from each individual mouse were assayed in triplicate. In the experiment in FIG. 5b normalization was done to β-cell number in each sample (pooled from 4 or more mice) scored by FACS following staining for insulin.

Tissue Section Staining and Analysis.

Tissue processing for tissue section staining was performed as previously described (Nir et al., Journal of Clinical Investigation, 2007, 117:2553-61), using the following antibodies: insulin (DakoCytomation), Ki67 (NeoMarkers RM9106S0), Pdx1 (gift from Christopher Wright, Vanderbilt University), Nlx6.1 (β-cell Biology Consortium), Chga (Novus NB120-15160) human p16 (Abcam ab108349 and BD Pharmingen 551153), p21 (Santa Cruz Biotechnology sc-397), Lamp2a (Abcam ab18528), pS6 (Cell Signaling 5364), E-cadherin (BD Pharmingen 610182), Atp5a (Abcam ab14748), Cox17 (Novus NBP1-19696) and Ndufb (Sigma-Aldrich HPA005640). Secondary antibodies were from Jackson ImmunoResearch Laboratories. Fluorescent images were taken on a Nikon C1 confocal microscope at 400× magnification. To calculate cell size on tissue sections, the circumference of E-Cadherin-stained cells was determined using Image J Software. >100 cells from 6 mice were scored for each group. Values shown are for p16-positive cells in p16-induced mice, and for β-cells in control mice. To determining expression levels of p16, pS6 and mitochondrial markers in stained islet sections of human subjects NIS-Elements software was used to measure the mean intensity per section area of the fluorescent signal within islets, divided by the mean intensity per area of the signal in the surrounding acinar tissue in the same field, to account for differential staining background. For β-cell area determination, consecutive paraffin sections 50 μm apart, spanning the entire pancreas, were stained for insulin and hematoxylin. Digital 40× images were obtained, stitched together using NIS-Elements software, and the fraction of tissue stained for insulin was determined.

FACS Analysis.

For flow cytometry, mouse islets were dissociated into a single-cell suspension with trypsin/EDTA treatment for 5 minutes at 37° C., followed by treatment with a cell fixation and permeablization solution (BD Pharmingen). Stained cells were analyzed on a MACSQuant Analyzer (DAKO). In all analyses cells were pooled from at least 3 mice in each group. Live human islets were obtained from pancreata of brain-dead patients as previously described (O'Gorman et al., Transplantation, 2010, 90:255-259), under approval of the Health Research Ethics Board of the University of Alberta and following patient informed consent. Patient details are presented in Tables 1 and 2. Several hundreds of islets were obtained from each subject, and were dissociated prior to staining. Antibody staining was performed using standard procedures, with the antibodies mentioned above, as well as: mouse p16 (Santa Cruz Biotechnology sc-1207), Lamp2a (Abcam ab18528). SA-βGal activity was assayed using the fluorescent β-galactosidase substrate C₁₂FDG (Invitrogen D-2893), as described (Debacq-Chainiaux et al., Nature Protocols, 2009, 4:1798-1806). 33 μM C₁₂FDG was added to dispersed islet cells and incubated for 1 hour at 37° C., with gentle shaking, and fluorescent cells were scored by FACS. For Mitotracker staining, 200 nM MitoTracker Green (Life Technologies) was added to dissociated islet cells and incubated for 1 hour at 37° C., with gentle shaking. Cells were washed once and visualized by FACS. For TMRE staining 7 nM tetramethylrhodamine ethyl ester perchlorate (Life Technologies) was added to dispersed islet cells and incubated for 1 hour at 37° C. in Krebs solution containing 3 or 20 mM Glucose, with gentle shaking. For the glucose uptake assay, 2-NBDG (Life Technologies N13195) was added to dispersed islet cells and incubated for 30 minutes at 37° C. in Krebs solution containing 3 mM Glucose, with gentle shaking.

Expression Profiling.

GFP-positive cells were isolated by FACS from dissociated islets of Insulin-rtTA/tet-GFP/tet-p16, and control Insulin-rtTA/Tet-GFP mice, following tet treatment for 10 days. Total RNA was isolated by TRIzol (Invitrogen) extraction followed by RNeasy Plus Micro Kit (Qiagen) from ˜50,000 β-cells, from 2 control and 3 p16-expressing mice. Libraries were prepared and sequenced using Illumina's directional RNA sequencing protocol (Hi Seq). Reads were mapped using TopHat2 and quantification and normalization were done using Cuffdiff to produce gene-level normalized expression values (FPKMs). Up- and downregulated genes with P<0.05 were tested for enrichment for gene sets using the hypergeometric method, FDR<0.05. Gene sets were derived from MSigDB or KEGG or compiled from the following literature: (Klochendler et al., 2016, Diabetes, 65(7):2081-2093; Stolovich-Rain et al., 2015, Developmental Cell, 32 (5): 535-545; Brady et al., 2011, Cell, 145 (4): 571-583; Boyer et al., 2006, Nature, 441 (7091): 349-353; Servitja et al., 2009, Molecular Cell Biology, 29 (11): 2945-59; Dorr et al., 2013, Nature, 501 (7467):421-426).

Human Islet mRNA.

Adult human islets for the quantitative RT-PCR were obtained from Integrated Islet Distribution program (http://iidp.coh.org/), and studied as described (Dai et al., Diabetologia, 2012, 55:707-718). Adult human islets were from 4 female and 5 male donors (age 44.7+4.2 years [range, 20-60], BMI 25.02+0.84 kg/m2 [range 21.2-29.1]. The cold ischemia time before pancreas isolation was 12.18+2.48 h. Nine normal juvenile pancreata were used in this study (6 months old, n=1; 14 months, n=1; 20 months, n=1; 3 years, n=2; 4 years, n=1; 5 years, n=2; 9 years, n=1) via protocol with the National Disease Research Interchange and International Institute for the Advancement of Medicine (IIAM). The islets were isolated as previously described (Walsh et al., Journal of Gastrointestinal Surgery, 2012, 16:1469-1477). De-identified human islet studies were approved by the Vanderbilt Institutional Review Board. Extraction of total RNA from human islets and performance of qRT-PCR were done as previously described (Dai et al., Diabetologia, 2012, 55:707-718), using the TaqMan primer-probe (CDKN2A, Hs00923894_m1) and reagents from Applied Biosystems (Foster city, CA). ACTB (Hs99999903_m1) was used as endogenous control.

Transmission Electron Microscopy.

Islets were fixed with 4% paraformaldehyde and 2.5% glutaraldehyde (EMS), post-fixed with 1% osmium tetroxide (Sigma), and dehydrated with increasing concentrations of ethanol followed by propylene oxide (Sigma). For embedding Agar 100 resin (Agar Scientific) was used. For imaging, 80 nm sections stained with 5% uranyl acetate for 10 min followed by 10 min with lead citrate were used. Samples were visualized with a transmission electron microscope (Technai 12, Phillips) equipped with a MegaView II CCD camera. To assess relative mitochondrial area, NIS elements were used.

Mitochondrial DNA Content.

To measure mitochondrial DNA copy number, DNA was isolated from sorted GFP-expressing β-cells by standard Phenol/Chloroform Extraction and Ethanol Precipitation. Quantitative real time PCR was used to evaluate the ratio between cytochrome b (mitochondrial) and L1 Repetitive element (nuclear). Cytochrome b: 5′-GCAGTCATAGCCACAGCATTT-3′ (SEQ ID NO:5) and 5′-AAGTGGAAAGCGAAGAATCG-3′ (SEQ ID NO: 6), L1: 5′-GTTACAGAGACGGAGTTTGGAG-3′ (SEQ ID NO: 7) and 5′-CGTTTGGATGCTGATTATGGG-3′ (SEQ ID NO: 8).

Oxygen Consumption.

Real-time measurements of mitochondrial oxygen consumption rate (OCR) were performed using the XF24 extracellular flux analyzer instrument and the AKOS algorithm (Seahorse). Islets were rinsed with sodium-bicarbonate-free DMEM supplemented with 0.5% BSA and 3 mM glucose, and 40 islets were distributed per well. After baseline measurements, the following were injected: glucose (20 mM), FCCP (5 μM), and rotenone and antimycin A (5 μM). The respiratory rate of each islet sample was measured at 37° C. and analyzed. Values in graph indicate mean of 5 replicates of 40 islets normalized to basal levels of oxygen consumption.

Western Blot.

Protein was extracted from fresh islets by RIPA lysis buffer supplemented with the protease and phosphatase inhibitors leupeptin, aprotonin and vanadate. Total protein was determined by Pierce BCA protein assay kit (Thermo scientific). Antibodies used were rabbit anti Pgc1α (Abcam ab54481) and rabbit anti Hsp90.

Human EndoC-βH2 Cells.

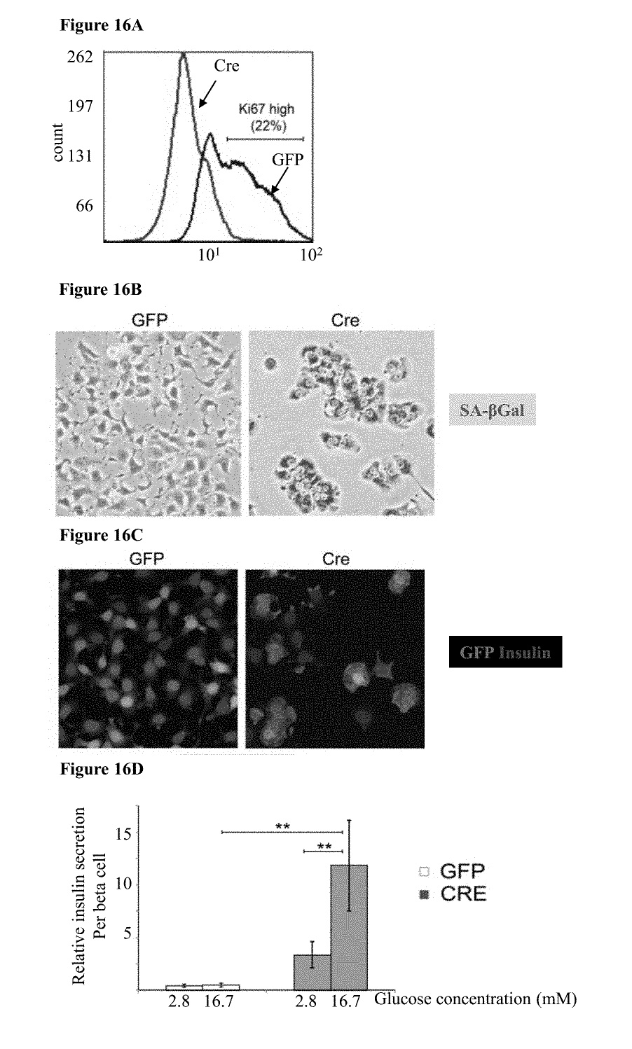

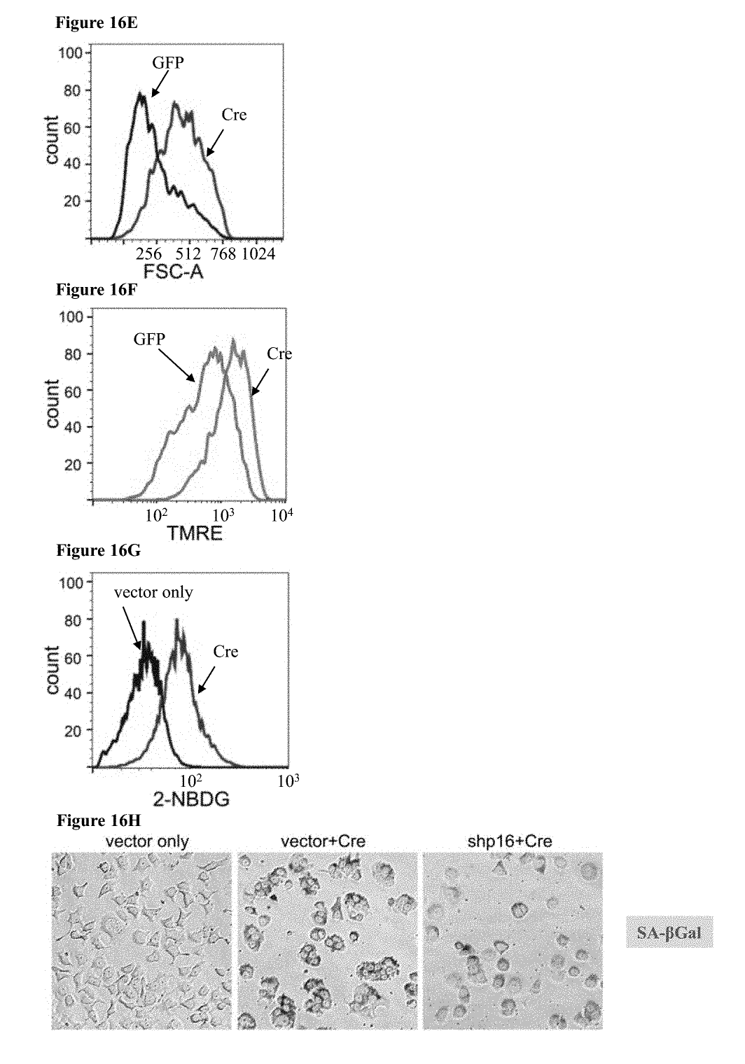

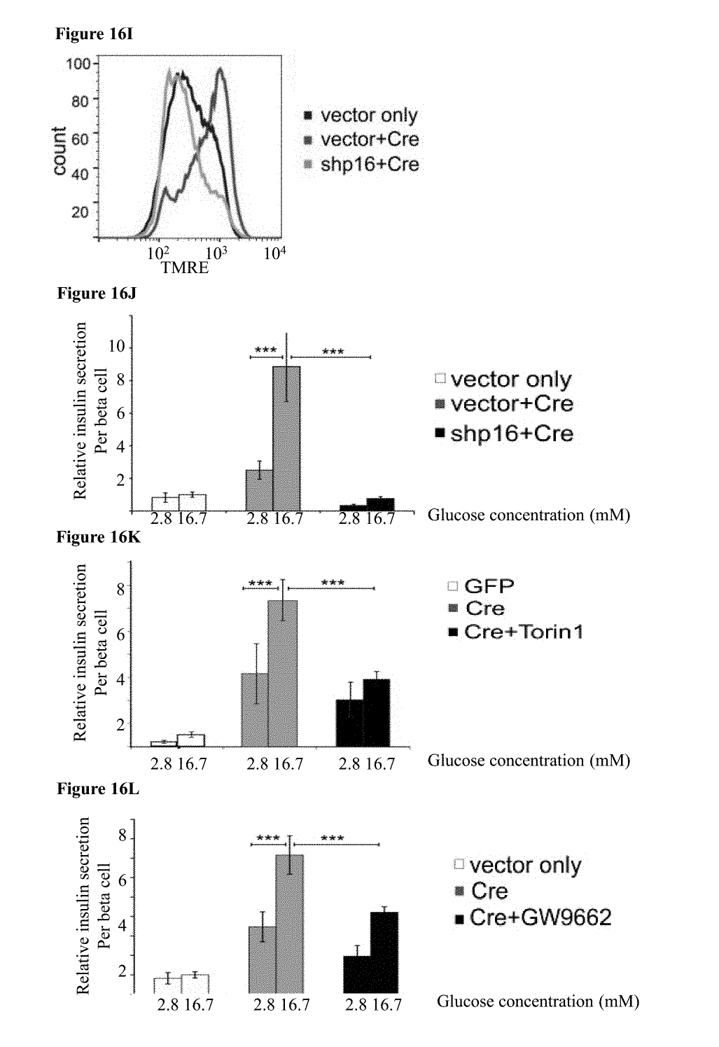

EndoC-βH2 cells were grown on Matrigel- and fibronectin-coated wells as previously described (Scharfmann et al., Journal of Clinical Investigation, 2014, 124:2087-98). Cells were infected with a lentivirus co-expressing Cre and GFP (Addgene #20781) or GFP only (pRRL-GFP); in FIGS. 16G-I, K Cre was expressed using the pTRIPdeltaU3-CMV-nlsCre vector, and the pLKO.1-Puro empty vector was used as a lentiviral infection control. For p16 silencing a pLKO.1-Puro-shp16 was used, carrying the targeting sequence: GCATGGAGCCTTCGGCTGACT (SEQ ID: 5). Viruses were produced by co-transfection of backbones into 293T cells with the packaging vectors pHRdelta8.2 and pCMV-VSV-G, media collection and centrifugal concentration. Cells were maintained following infection for 3 weeks. SA-βGal and TMRE stains as well as cell size and insulin secretion were performed 3 weeks after infection using the same methods as above. For GSIS cells were plated 3 weeks after infection in 96-well plates at 3.5×10⁴ cells/well for control cells and 7×10⁴ cells/well for Cre-expressing cells. Seven days later, cells were incubated overnight in culture medium that contained 2.8 mM glucose and then in Krebs buffer containing 2.8 mM glucose for 60 minutes. Media was collected, and the cells were then incubated in Krebs buffer containing 16.7 mM glucose for 60 minutes, and media collected again. Insulin levels in media were measured in 12 replicates per group by ELISA. Values were normalized to cell number in each well. For mTor inhibition, the cells were treated with 250 nM Torin1 starting 3 days after infection, and maintained in Torin1 for the duration of the experiment. For PPARγ inhibition cells were treated with GW9662 50 mM (Sigma) twice a week for the duration of the experiment.

### Example 1: p16 Induces Cell-Cycle Arrest and Senescence in Pancreatic β-Cells

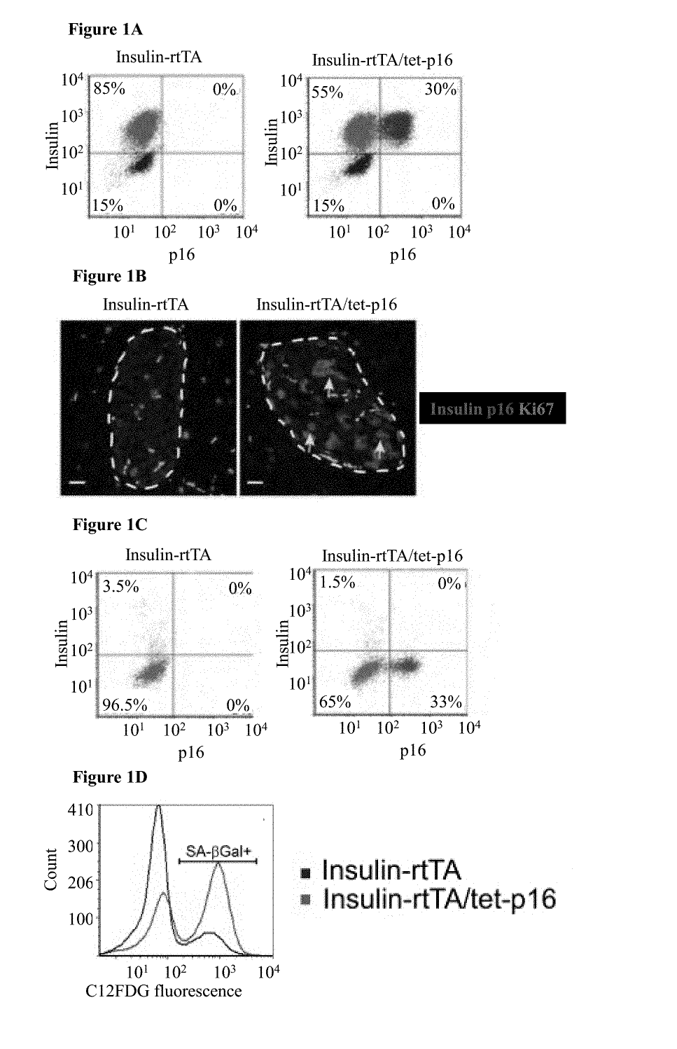

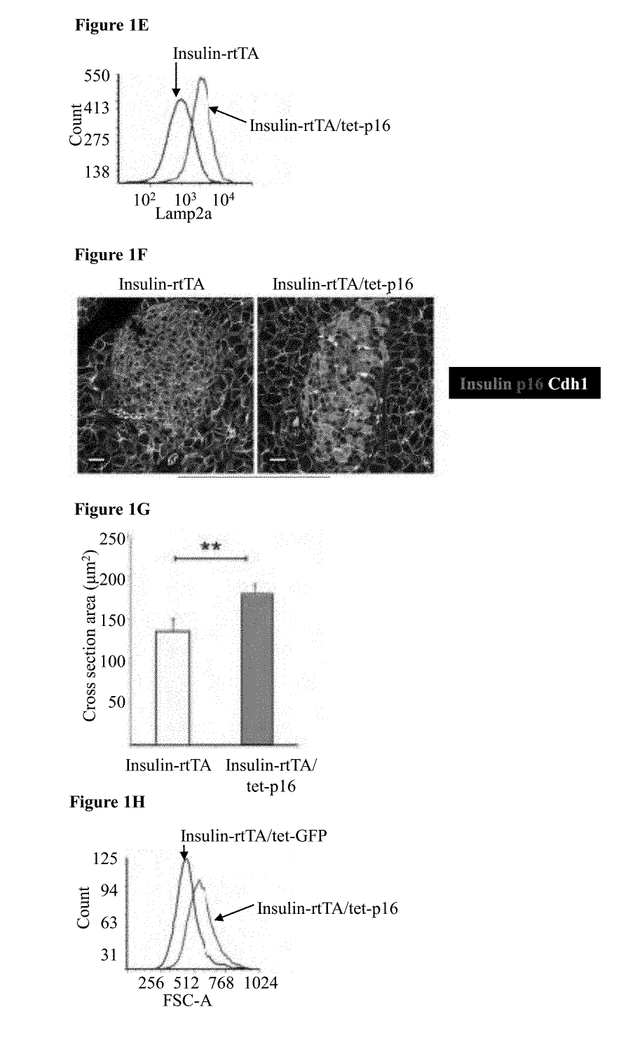

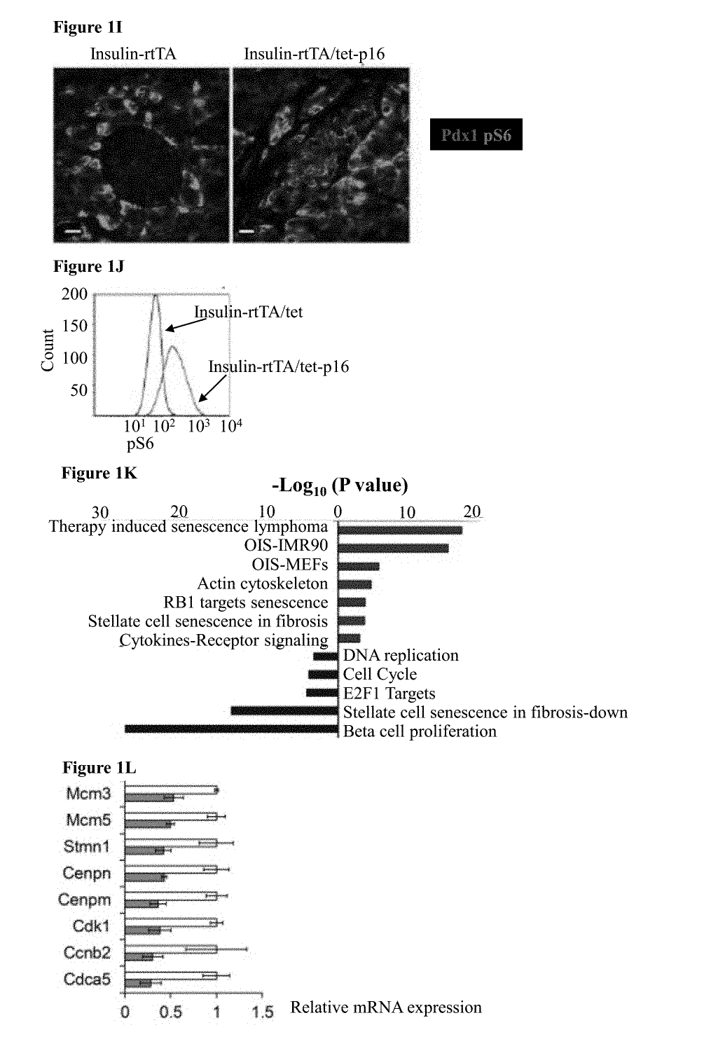

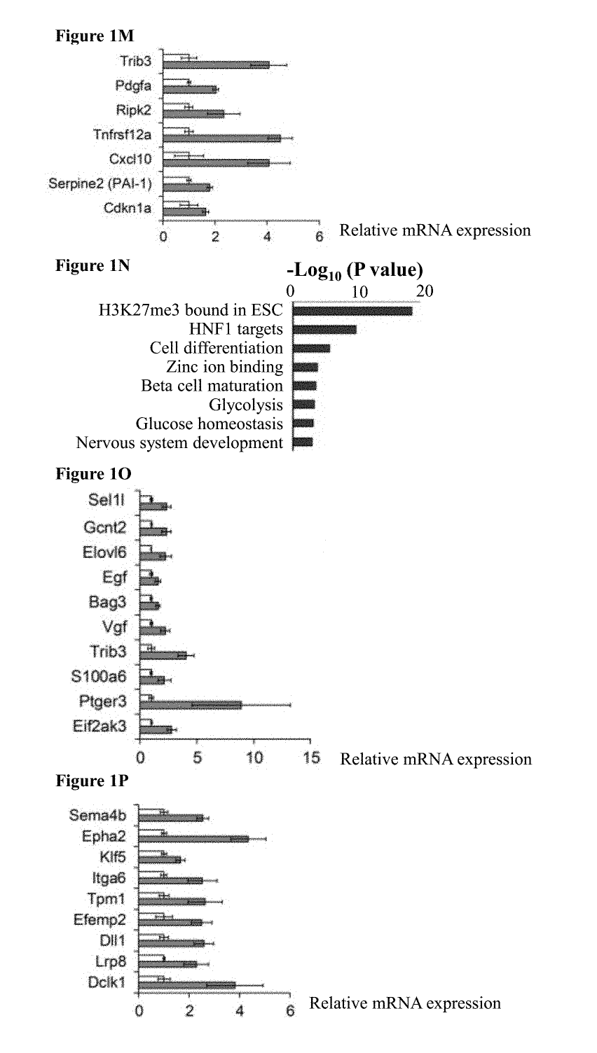

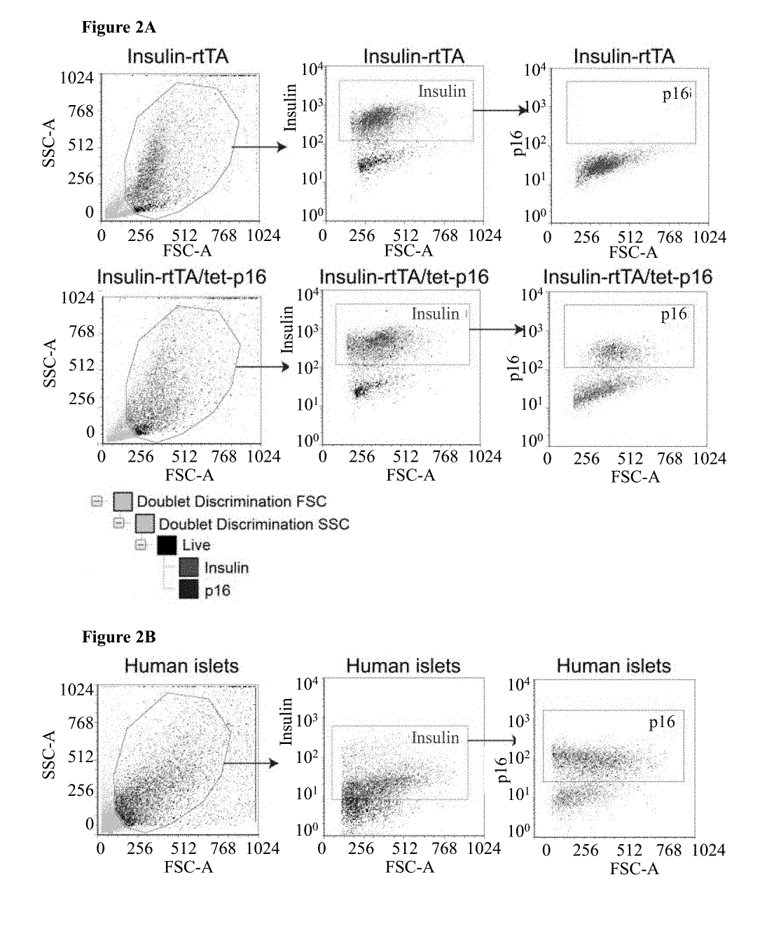

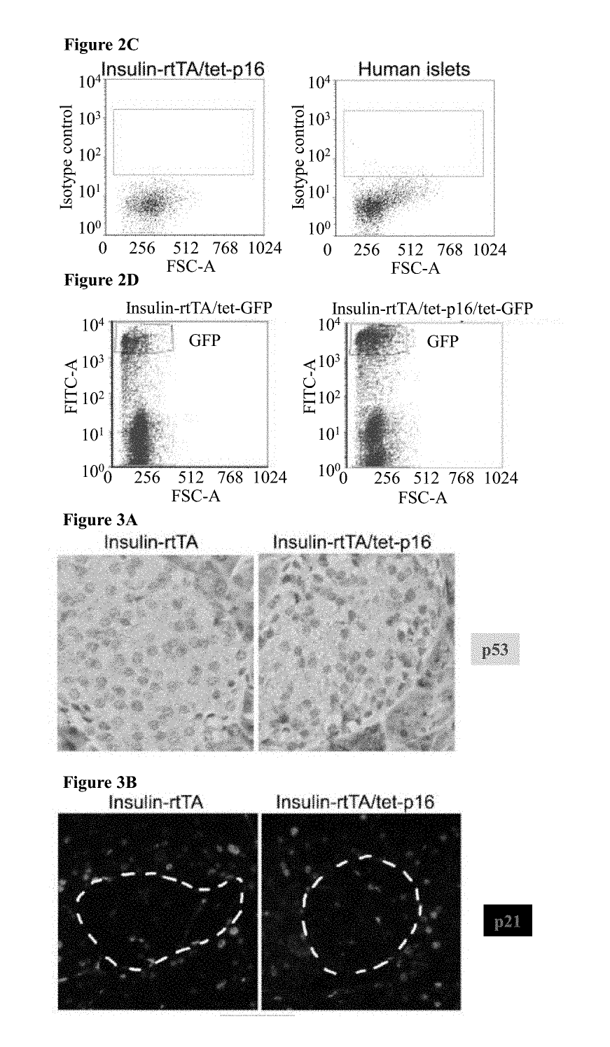

To study the effects of p16 expression in β-cells mice carrying a tetracycline-inducible human p16 transgene (tet-p16) and crossed them with mice expressing the reverse tetracycline-dependent transactivator under control of the insulin II promoter (Insulin-rtTA) were generated, thus allowing β-cell specific p16 activation upon tetracycline (tet) treatment. In mice treated for 10 days starting at 3-4 weeks of age, transgenic p16 was detected by immunostaining in ˜35% of β-cells; lower-level expression in additional cells could not be excluded (FIGS. 1A-B and FIG. 2A, C). Ki67 staining revealed that 3.5% of β-cells in control mice were proliferating, whereas p16-expressing β-cells were non-proliferative (FIG. 1B-C). Senescence-associated β-galactosidase (SA-βGal) activity and the expression of the lysosomal marker Lamp2a were increased in p16-expressing islets, indicating that p16 induces features of senescence (FIG. 1D-E). Strikingly, p16-expressing β-cells were approximately 1.3-fold larger than control β-cells, as measured by both FACS and image analysis (FIG. 1F-H, FIG. 2B-C). Consistent with this observation, islets of p16-expressing mice had higher levels of phosphorylated ribosomal protein S6 (rpS6), an mTOR target and regulator of β-cell size (FIG. 1I-J). While enlarged cell-volume is a central feature of senescent cells in culture, there have been few, if any, reports of this phenotype in senescence occurring in vivo.

### Example 2: p16 Expressing β-Cells Express Senescence Genes and Genes Associated with Functional Maturation

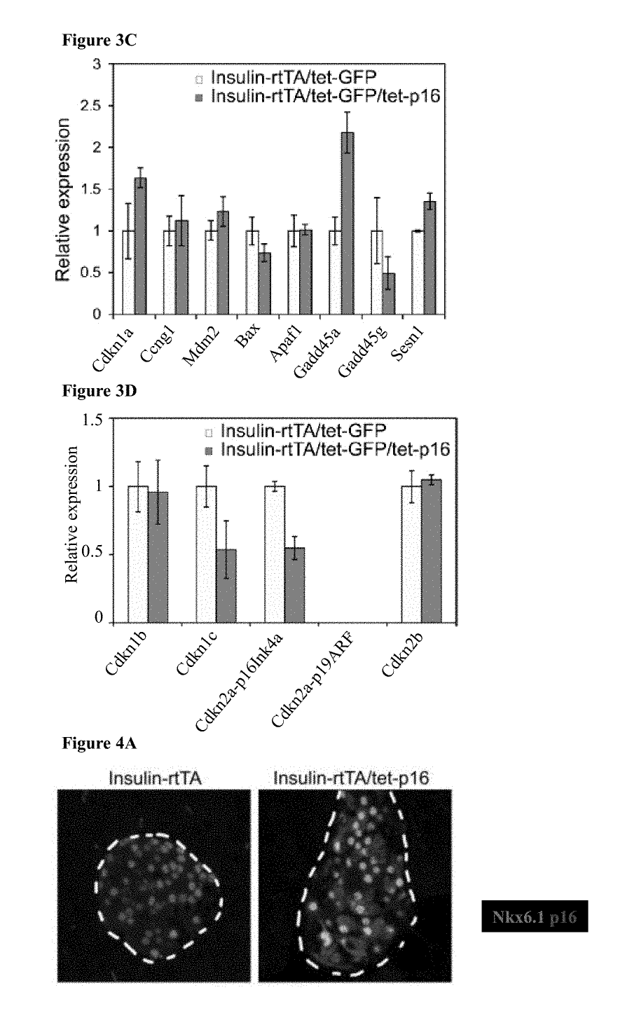

To isolate a population of β-cells enriched for p16 expression, triple-transgenic mice carrying a tet-GFP transgene in addition to tet-p16 and Insulin-rtTA were generated, such that GFP and p16 are co-activated in a largely overlapping subset of β-cells. The mice were treated with tet for 10 days, their islets were dissected and dissociated, and GFP-positive cells were isolated by FACS (FIG. 2C-D). The transcriptome of these cells was compared to that of control β-cells isolated from mice expressing only the GFP transgene (FIG. 2D). It was found that genes associated with cell proliferation were suppressed in p16-expressing β-cells; in contrast, gene sets previously shown to be elevated in senescent cells were significantly upregulated by p16 expression (FIG. 1K-M). Strikingly, these sets were derived from diverse biological settings, including chemotherapy-treated lymphoma cells, Ras-expressing mouse and human fibroblasts, and fibrotic liver stellate cells. Gene sets associated with cytoskeletal structure and interaction with the ECM, changes in which are typical to senescence, were also upregulated, as was a group of genes encoding secreted proteins (FIG. 1K). The p53 target p21^{cip1} (Cdkn1a) was mildly upregulated, as was the senescence marker and p53 target PAI1 (SerpinE2) (FIG. 1L), yet other p53 targets were not significantly activated (FIGS. 3B-D), and p53 protein was not detected in islet sections (FIG. 3A), indicating limited activation of this pathway. Thus, p16 is sufficient to activate hallmarks of the senescence gene expression program in β-cells.

Further data concerning genes that are up and down regulated by p16 expression can be found in the following article, Helman et al., 2016, Nature Medicine, 22(4):412-420, which is hereby incorporated by reference.

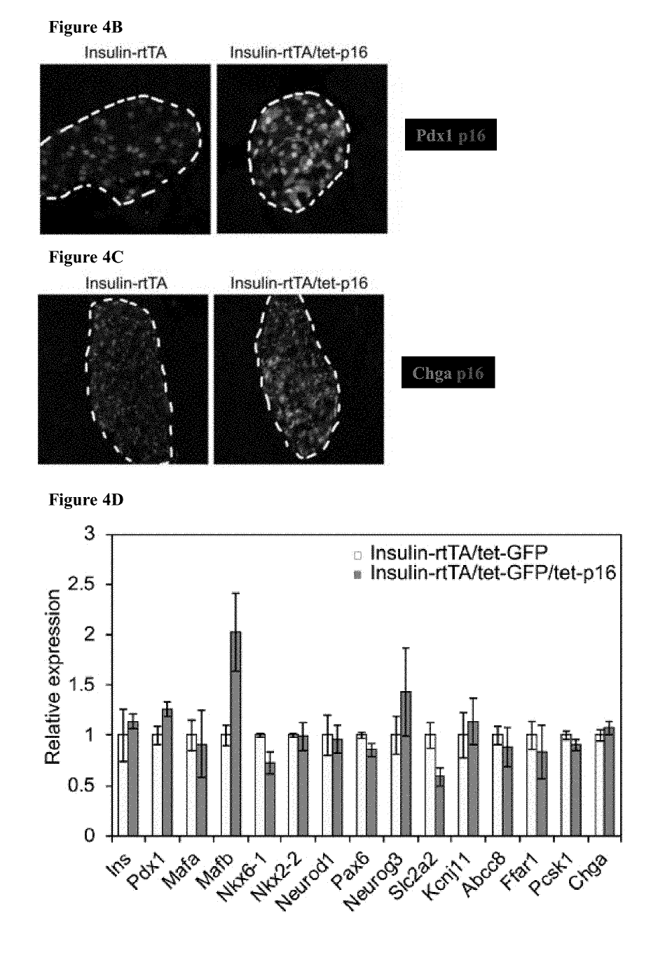

Central makers and regulators of β-cell differentiation were minimally changed upon p16 expression (FIG. 4A-D), indicating that the cells maintained their identity. However, upregulation of genes whose expression increases in adult versus neonatal β-cells was observed, in correlation with the acquisition of functionality (FIG. 1N-O). Genes repressed by the Polycomb complex were preferentially upregulated in p16-expressing β-cells (FIG. 1N), a feature typically associated with differentiation and β-cell aging, as were other sets associated with β-cell maturation, including targets of HNF1, and nervous-system genes, many of which were Polycomb targets (FIG. 1N, P).

Together these findings suggested that p16 enhances aspects of β-cell maturation in conjunction with activation of the senescence program.

### Example 3: p16 Increases Glucose-Stimulated Insulin Secretion

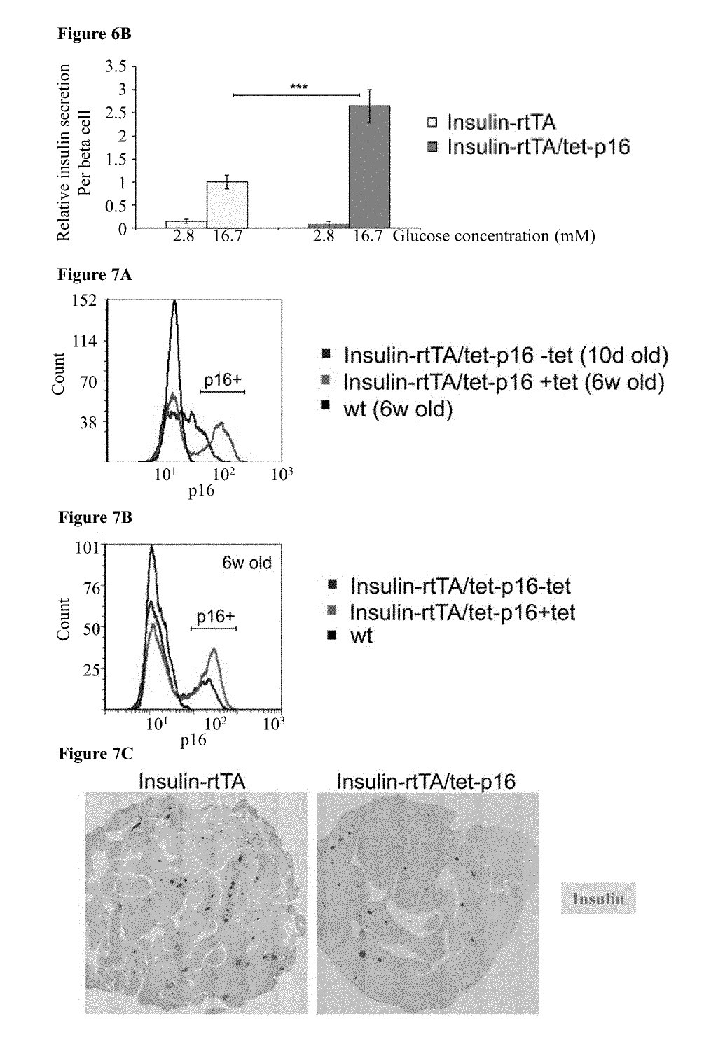

To directly study the effect of p16 expression on β-cell function, pancreatic islets were isolated from p16-expressing and control mice following 10 days of tet treatment starting at 4 weeks of age, and measured glucose stimulated insulin secretion (GSIS). While the levels of insulin secreted from islets at low glucose did not significantly differ between p16-expressing and control islets, p16-expressing islets secreted approximately 2.5-fold more insulin upon glucose stimulation (FIG. 5A). This increase was evident when secreted insulin levels were normalized either to total insulin or to protein content in the islet samples (FIG. 5A and FIG. 6A). Normalization of insulin levels directly to β-cell number, scored by FACS, also indicated that secretion per β-cell was elevated (FIG. 5B). GSIS remained high in p16-expressing islets after two months of p16 induction, indicating that this represented a stable phenotypic change (FIG. 5C). Activation of p16 in sexually mature mice, at 6 weeks of age, similarly elevated GSIS (FIG. 6B). The insulin content of p16-expressing β-cells was approximately 1.5-fold higher than that of control β-cells (FIG. 5D).

These findings reveal that p16-induced senescence increases the capacity of β-cells to release insulin following glucose stimulation.

### Example 4: p16 Overexpression Improves Glucose Homeostasis in a Murine Model of Diabetes

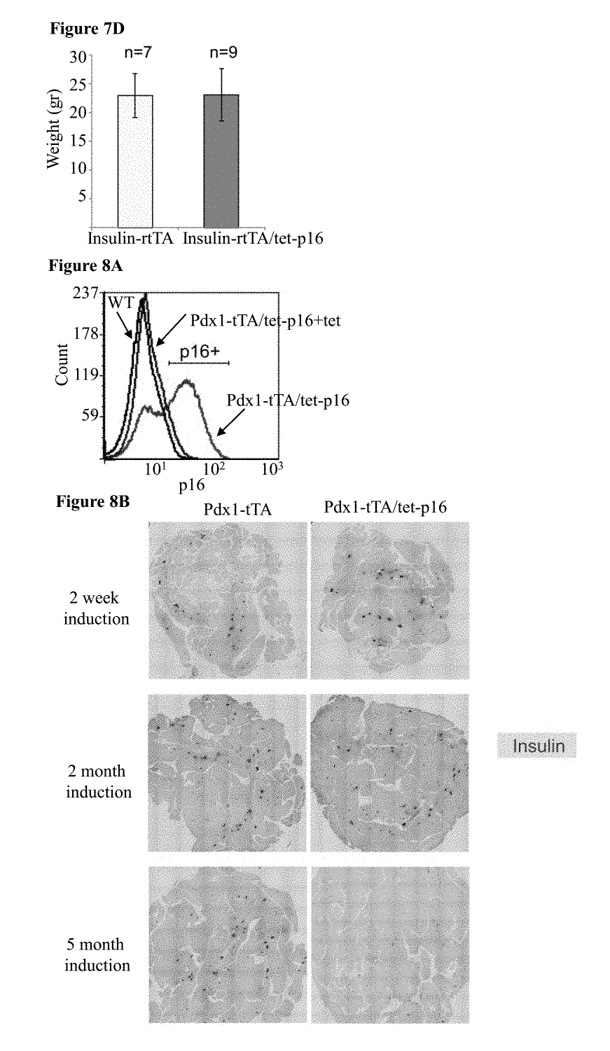

Improved β-cell function could be expected to alter physiological glucose tolerance. Insulin-rtTA/tet-p16 mice were, however, not suitable for testing this hypothesis, since they suffered from decreased overall β-cell mass due to low tetracycline-independent p16 expression (FIG. 7A-D). Therefore, the effects of p16 induction were tested on Pdx1-tTA mice, which are deficient in one allele of the Pdx1 transcription factor, mimicking a monogenic familial form of diabetes, MODY4 (Holland et al., 2002, Protocols of the National Academy of Science, 99:12236-41). These mice were particularly suitable for this analysis since their hyperglycemia is rooted in impaired β-cell function rather than systemic causes, and, in addition, when crossed to tet-p16 mice, allowed tightly regulated β-cell-specific inducible gene activation (FIG. 8A).

p16 was activated in 1 month-old Pdx1-tTA/tet-p16 mice for 2 weeks. Glucose tolerance was significantly improved in these mice compared to the diabetic Pdx1-tTA mice, indicating that p16 partially reverses the impaired function of β-cells in this model (FIG. 5E). Serum insulin levels following glucose injection were significantly elevated in Pdx1-tTA/tet-p16 mice, accounting for their improved glucose tolerance (FIG. 5F). p16-expressing β-cells were larger, reversing the decreased cell size observed in Pdx1-tTA mice relative to wild-type mice (FIG. 5G). Sensitivity to insulin was not improved in p16-expressing mice and mouse body weights were unchanged (FIG. 511 and FIG. 8B), consistent with changes in β-cell function being the primary cause of improved glucose tolerance.

Following 2 months of p16 induction, Pdx1-tTA/tet-p16 mice still displayed improved glucose tolerance, indicating that the p16-induced enhancement of β-cell function was a stable phenotype (FIG. 51). However, following 5 months of induction glucose tolerance in these mice deteriorated to the level of Pdx1-tTA mice (FIG. 5J). At this time, β-cell mass was reduced, likely due to reduced β-cell proliferation, accounting for the decline in glucose tolerance (FIG. 5K and FIG. 8C). Together these findings revealed that p16 expression in β-cells induces a substantial physiologic improvement in glucose tolerance through increased insulin secretion, which can rapidly counter mild hyperglycemia; however, this comes at the expense of β-cell proliferative potential, and therefore can lead to reduced β-cell mass and functional deterioration over the long term.

### Example 5: Increased Glucose Uptake and Mitochondrial Activity in p16-Induced β-Cells

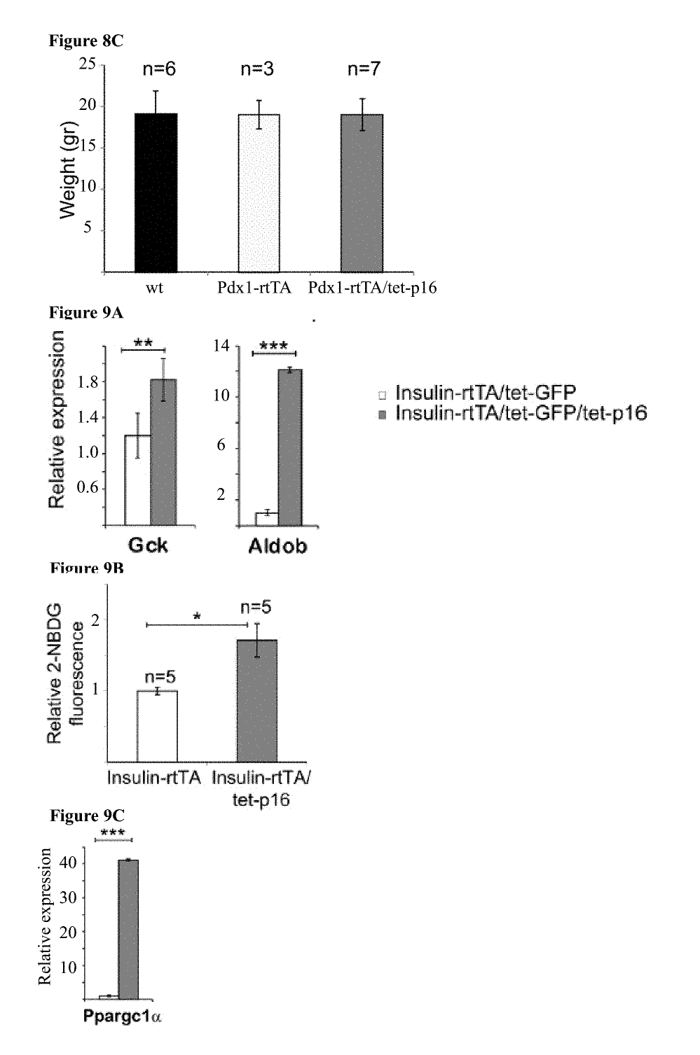

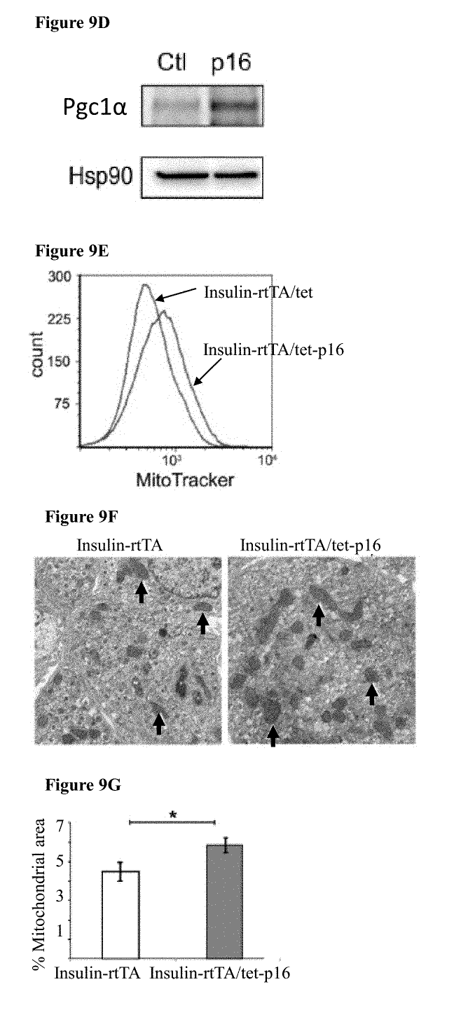

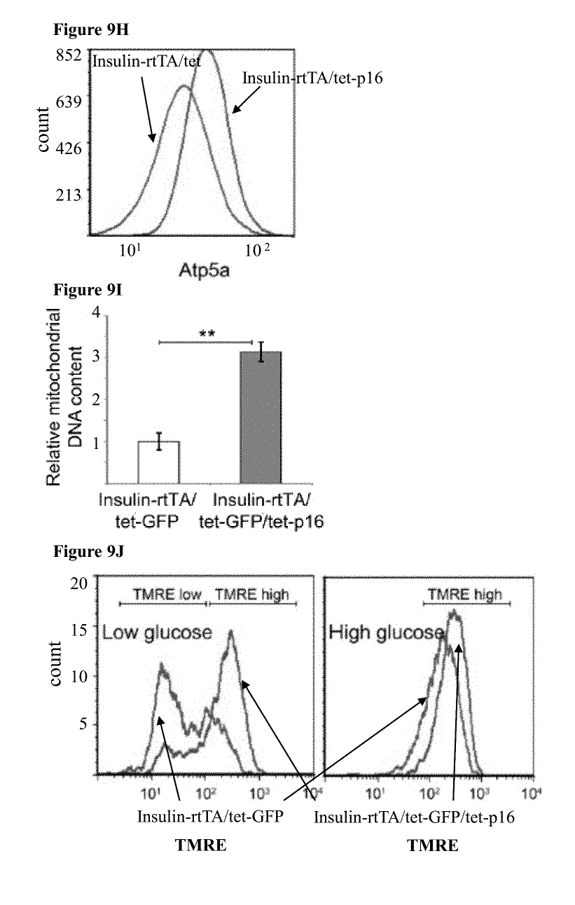

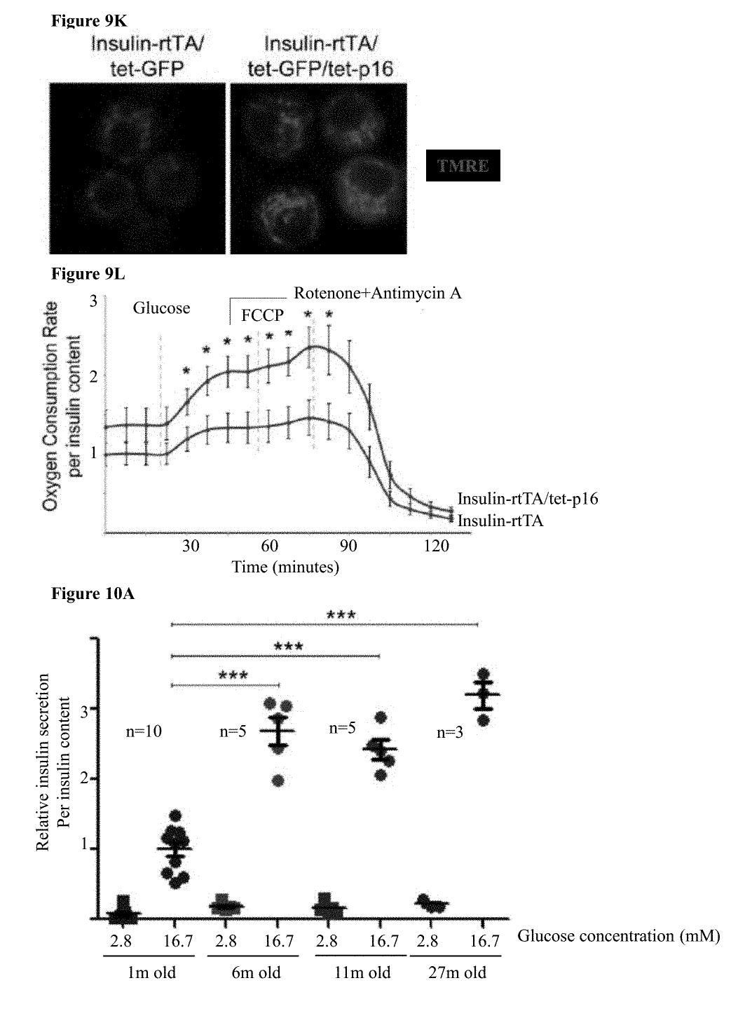

Next studied was the cellular mechanisms underlying enhanced insulin secretion upon p16 induction. Insulin secretion is primarily controlled by oxidative metabolism of glucose in the mitochondria: increased ATP levels lead to membrane depolarization, calcium influx and release of insulin. Recent studies have shown that cellular senescence of different cell types involves enhancement of glucose uptake, glycolysis and oxidative phosphorylation. Examination of the transcriptome of p16-expressing β-cells revealed increased expression of gene sets associated with glucose metabolism (FIG. 1N). Among the upregulated genes were aldob, encoding the glycolytic enzyme fructose-bisphosphate aldolase B, and the gene gck, encoding glucokinase, the β-cell-specific hexokinase, which controls glucose uptake and glycolysis rates (FIG. 9A). Therefore, glucose uptake by islet cells of p16-expressing Insulin-rtTA/tet-p16 mice was measured, and an increase of approximately 1.7-fold relative to controls was found (FIG. 9B).

The levels of Pgc1α, the master transcription factor controlling mitochondrial biogenesis, were also dramatically elevated in p16-expressing β-cells (FIGS. 9C-D). It was found that, indeed, p16-expressing β-cells contained increased numbers of mitochondria, as evident by electron microscopy analysis, staining of mitochondrial markers, and quantification of mitochondrial DNA content (FIGS. 9E-F). Furthermore, mitochondrial membrane potential, indicating mitochondrial activity, was increased in p16-expressing β-cells (FIGS. 9J-K), and p16-expressing islets consumed more oxygen in basal conditions and in response to glucose stimulation than control islets (FIG. 9L). Together, these findings indicate that p16 expression in β-cells leads to increased glucose uptake, mitochondrial biogenesis and mitochondrial respiration, providing a direct link to consequent increased GSIS.

### Example 6: Endogenous p16 Activity During Normal Mouse Maturation Enhances Insulin Secretion

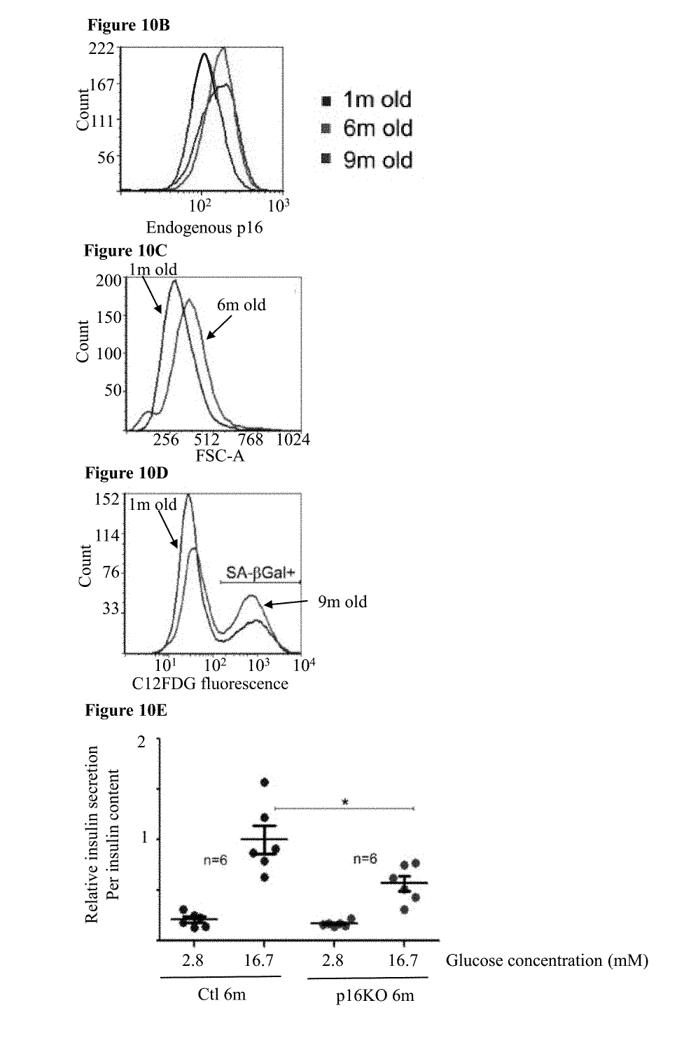

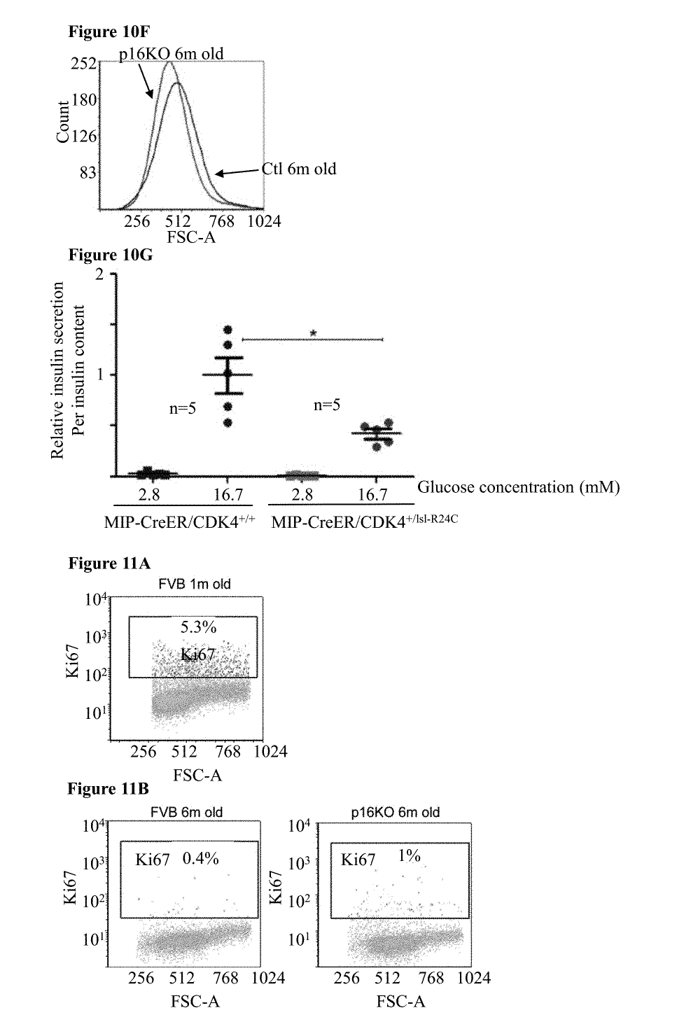

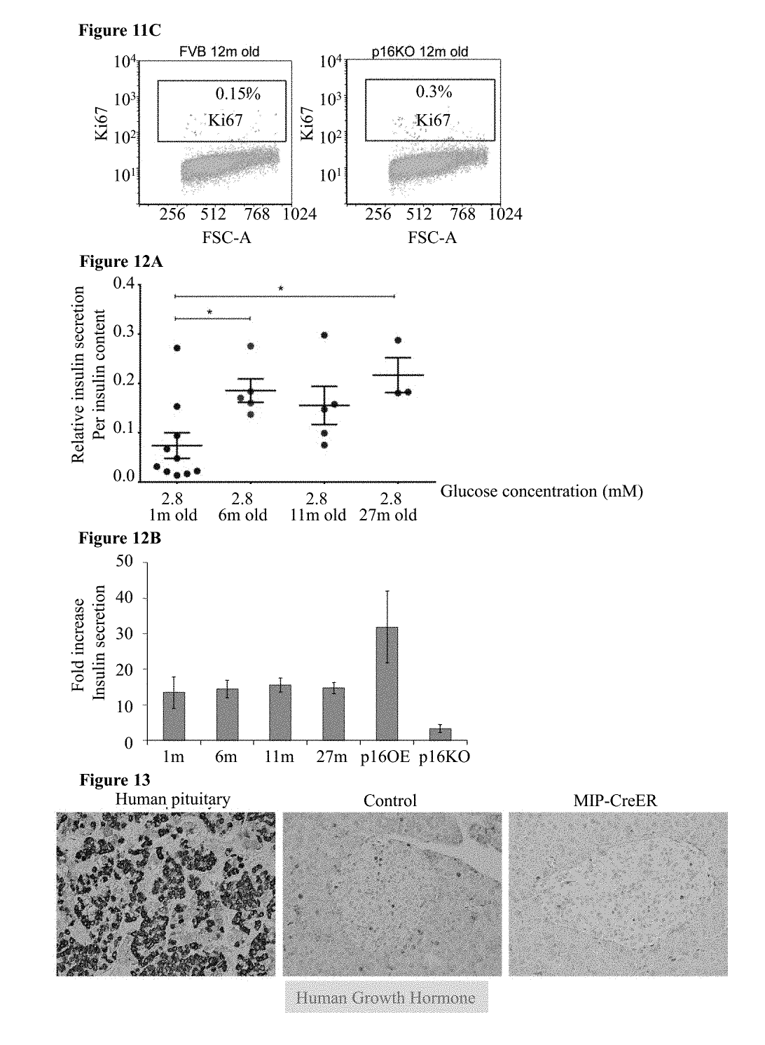

It was next asked whether endogenous p16, whose expression accumulates in β-cells with age, influences insulin secretion capacity. Islets isolated from wild type mice at 6 and 11-months of age (mature), and at 27-months (geriatric), showed higher GSIS than 1-month-old (juvenile) mice, concomitant with their expression of endogenous p16 (FIG. 10A-B). β-cells of mature mice showed reduced proliferation, were larger, and exhibited higher SA-βGal activity (FIGS. 10C-D and FIG. 11A). This indicated that GSIS in fact increases with age. Notably, in contrast to p16 transgenic mice, islets of mature and geriatric mice showed elevated insulin secretion also at low glucose levels, and therefore the fold-change in insulin secretion was not changed relative to juvenile mice (FIG. 10A and FIG. 12A-B).

To study whether p16 contributes to these maturation-associated changes, the pancreata of mature p16-null mice were examined. As previously reported (Krishnamurthy et al., 2006, Nature, 443: 453-457), the number of proliferating β-cells was more than 2-fold higher in 6 and 12 month-old p16-null mice than in wild-type counterparts (FIG. 11B-C). However, islets of mature p16-null mice secreted less insulin upon glucose stimulation than did islets of age-matched control mice, and their β-cells were smaller (FIG. 10E-F). This finding indicates that endogenous p16 expression during normal aging and maturation drives enhanced GSIS capacity.

Next examined was whether p16 drives changes in β-cell function through its central well-established function, inhibition of CDK4. It was tested whether β-cell-specific expression of a constitutively active allele of CDK4 will lead to reduced GSIS, similar to p16 ablation. To do this mice carrying a conditional knocked-in CDK4 allele carrying the R24C activating mutation (CDK4^{+/lsl-R24C})³⁵ were crossed with mice expressing a tamoxifen-inducible Cre under the control of the mouse insulin promoter (MIP-CreER) (FIG. 13). The CDK4^{R24C} allele in β-cells was activated by tamoxifen injection, and collected pancreatic islets 2 weeks subsequently. CDK4^{+/R24C} islets displayed reduced GSIS relative to matched control islets, recapitulating the effects of p16 deficiency (FIG. 10G). This finding supports the hypothesis that p16 acts through CDK4 inhibition to enhance GSIS.

### Example 7: β-Cell Senescence in Human Islets

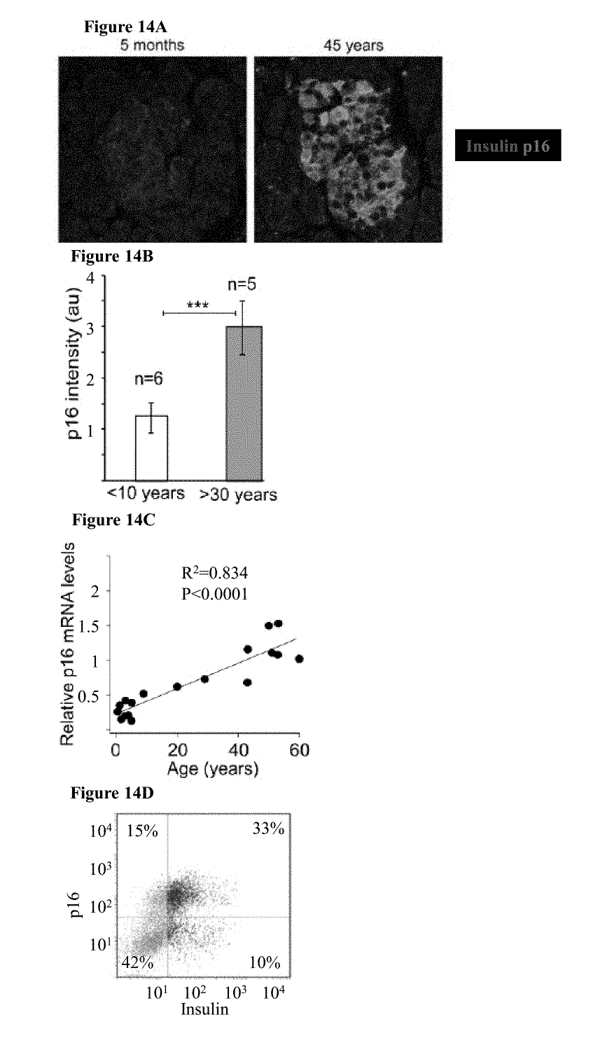

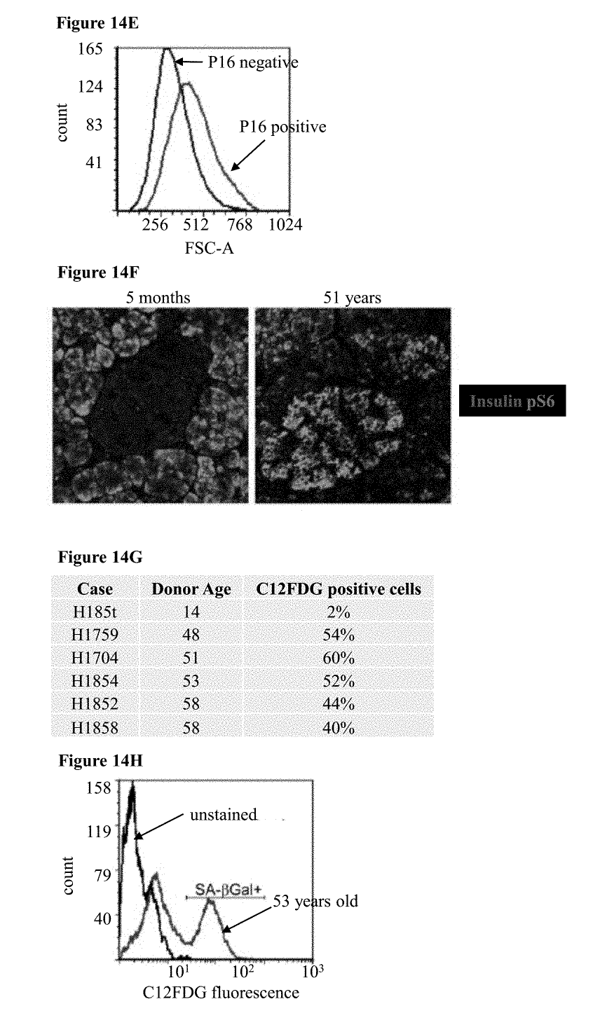

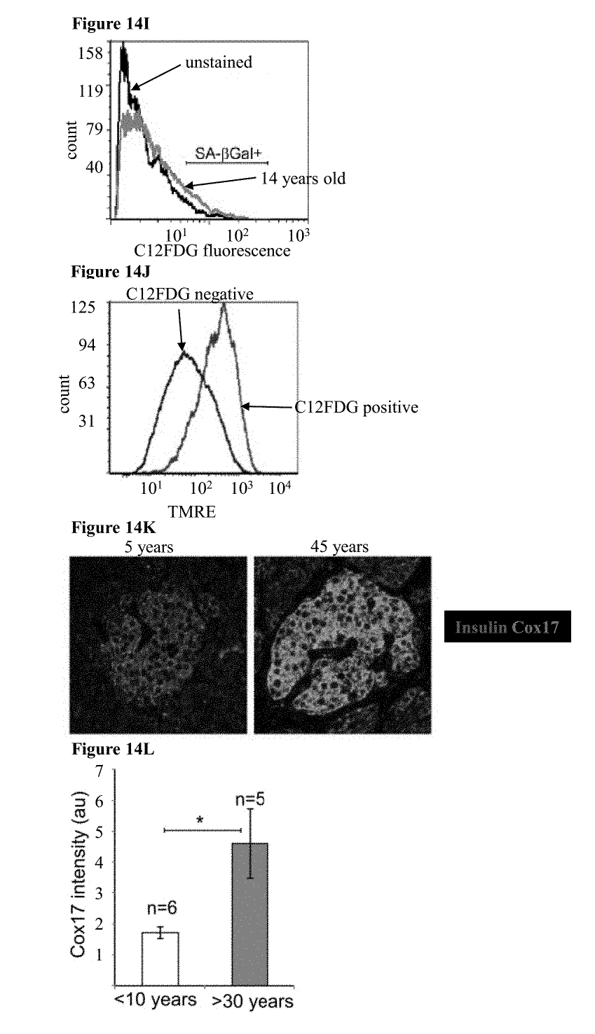

It was next tested whether β-cell senescence occurs in humans. Immunostaining of sections of human pancreata and mRNA analysis of human islets, revealed elevated p16 expression with age (FIGS. 14A-C and Tables 1 and 2). Human islets obtained live from recently-deceased middle aged human subjects were dissociated, and co-stained the cells for p16 and insulin (FIG. 14D and FIG. 2B). p16-expressing β-cells had larger mean volumes than p16-negative β-cells from the same islets (FIG. 14E). Levels of phospho-S6 staining in islets also increased with age, as observed in p16 transgenic mice (FIG. 14F and FIG. 13), consistent with the senescence-associated changes observed in mice.

TABLE 1
List of analyzed human islet sections used for immunostaining.

| | Donor | AutoAb | Age | | | | |
|---|---|---|---|---|---|---|---|
| CaseID | Type | (RIA) | (years) | Gender | Ethnicity | BMI | Analysis |
| 6117 | No | Negative | 0.33 | Male | Caucasian | 18.4 | Immunostaining p16 |
| | diabetes | | | | | | Cox17 Atp5a pS6 |
| 6125 | No | Negative | 0.42 | Male | Caucasian | 18.9 | Immunostaining p16 |
| | diabetes | | | | | | Cox17 Ndufb5 |
| 6122 | No | Negative | 0.42 | Female | Caucasian | 13.8 | Immunostaining p16 |
| | diabetes | | | | | | Cox17 pS6 |
| 6107 | No | Negative | 2.2 | Male | African | 15.9 | Immunostaining p16 |
| | diabetes | | | | American | | Cox17 |
| 6005 | No | Negative | 5 | Female | Caucasian | 15.7 | Immunostaining p16 |
| | diabetes | | | | | | Cox17 pS6 |
| 6144 | No | Negative | 7.5 | Female | Hispanic | 16.3 | Immunostaining p16 |
| | diabetes | | | | | | Cox17 |
| 6034 | No | Negative | 32 | Female | Caucasian | 25.2 | Immunostaining p16 |
| | diabetes | | | | | | Cox17 pS6 |
| 6009 | No | Negative | 45 | Male | Caucasian | 30.6 | Immunostaining p16 |
| | diabetes | | | | | | Cox17 Atp5a pS6 |
| 6165 | No | Negative | 45.8 | Female | Caucasian | 25 | Immunostaining p16 |
| | diabetes | | | | | | Cox17 pS6 |
| 6168 | No | No serum | 51 | Male | Hispanic | 25.2 | Immunostaining p16 |
| | diabetes | available | | | | | Cox17 Ndufb5 pS6 |

TABLE 2
List of analyzed human islet used for the FACS and analyses.

| | Age | | |
|---|---|---|---|
| CaseID | (years) | Gender | Analysis |
| H1744 | 56 | Female | FACS Insulin p16 |
| H1704 | 51 | Male | FACS C12FDG |
| H1798 | 49 | Female | FACS p16 Lamp2a |
| H1759 | 48 | Male | FACS C12FDG |
| H1785 | 42 | Male | FACS Insulin p16 |
| H1851 | 14 | Male | FACS C12FDG |
| H1852 | 58 | Female | FACS C12FDG TMRE |
| H1854 | 54 | Female | FACS C12FDG TMRE |
| H1858 | 57 | Male | FACS C12FDG TMRE |

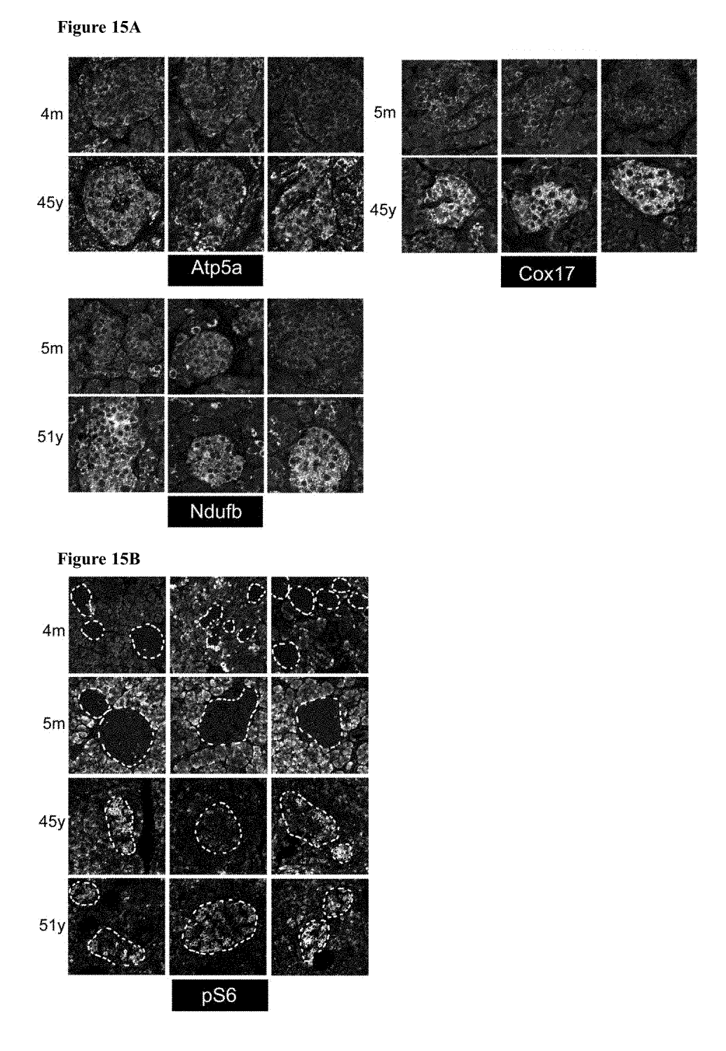

To directly detect senescence, live dissociated human islet cells were stained for SA-βGal. Samples obtained from middle-aged donors all contained a substantial SA-βGal-positive fraction (40-60% of cells, mean=50±8% S.D.) indicating the presence of senescent β-cells in these islets (FIG. 14G-H). While live cadaver material from young subjects is rare, one sample, from a 14 year-old subject was obtained; interestingly, SA-βGal-positive cells were not detected in this sample (FIG. 14G, I). Mitochondrial activity in the SA-βGal-positive and SA-βGal-negative cell fractions was compared by TMRE staining. Strikingly, stronger TMRE was observed in the SA-βGal-positive cells, indicating increased mitochondrial activity in senescent β-cells (FIG. 14J). Consistent with this, pancreas sections of middle-aged subjects expressed increased levels of mitochondria-specific proteins relative to young subjects (FIG. 141, K and FIG. 15A-B). Together, these findings indicate that during human aging p16-expressing senescent β-cells accumulate in islets, and feature increased size and mitochondrial numbers and activity.

### Example 8: Increased Insulin Secretion by Senescent Human β-Cells

Next, whether senescence leads to increased insulin secretion in human cells was examined. EndoC-βH2 cells are a line of fetal pancreas-derived cells that was recently successfully propagated through introduction of the SV40 Large-T-antigen and hTERT. Cre-mediated excision of these immortalizing genes, which allows re-activation of Rb and p53, was shown to cause these cells to cease dividing and to increase insulin expression and secretion. Since Rb and p53 represent the central mediators of senescence, whether in fact Cre-mediated removal of T-antigen led to senescence of these cells was tested. It was found that EndoC-βH2 cells infected with a Cre-expressing lentivirus acquired a clear senescent morphology and displayed high SA-βGal activity, relative to the same cells infected with a GFP-expressing virus (FIG. 16A-B). Indeed, this was accompanied by a dramatic elevation in insulin secretion capacity (FIG. 16C-D). Consistent with our findings in mouse and human islets, the senescent EndoC-βH2 cells were larger, and displayed enhanced glucose uptake and mitochondrial activity relative to control cells (FIG. 16E-G). This finding indicated that senescence leads to enhanced GSIS in human pancreatic cells.

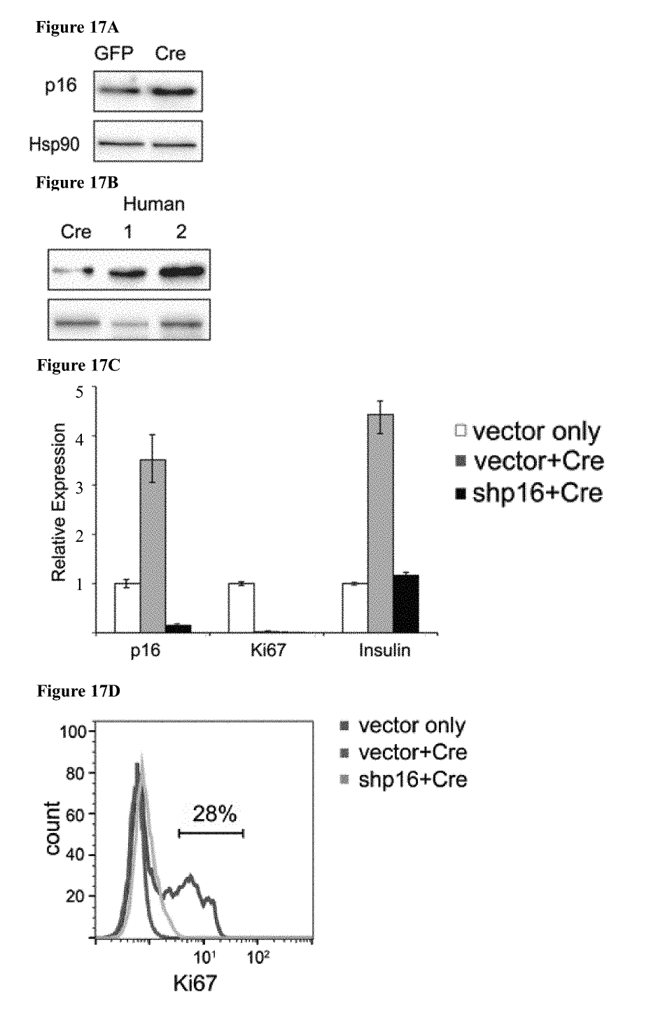

It was next tested whether p16 drives senescence and enhanced GSIS in the EndoC-βH2 cells. It was found that endogenous p16 was expressed in the cells prior to introduction of Cre, and was further elevated subsequent to it (FIG. 17A-B). Therefore, EndoC-βH2 cells in which p16 was stably silenced using lentiviral shRNA were generated, and then infected with a Cre-expressing virus (FIG. 17C). p16-silenced cells showed reduced levels of SA-βGal following Cre expression relative to cells expressing a control vector, yet still stopped dividing (FIG. 16H and FIG. 17C-D). Strikingly, p16 silencing completely abolished the acquisition of GSIS capacity and of increased mitochondrial activity following Cre expression (FIGS. 16I-J and FIG. 17C). This indicates that p16 is essential for induction of senescence of these human cells and the associated enhanced GSIS; upon release of Rb from inhibition by Large-T-antigen, its ability to induce these effects is apparently highly dependent on the activating signal from p16. This finding also further indicates that p16 acts through the CDK4-Rb pathways and the senescence machinery: its ability to induce senescence and GSIS was dependent on the release of Rb (and p53) from inhibition by Large-T-antigen.

### Example 9: mTOR and PPARγ Contribute to Enhanced GSIS Upon Senescence

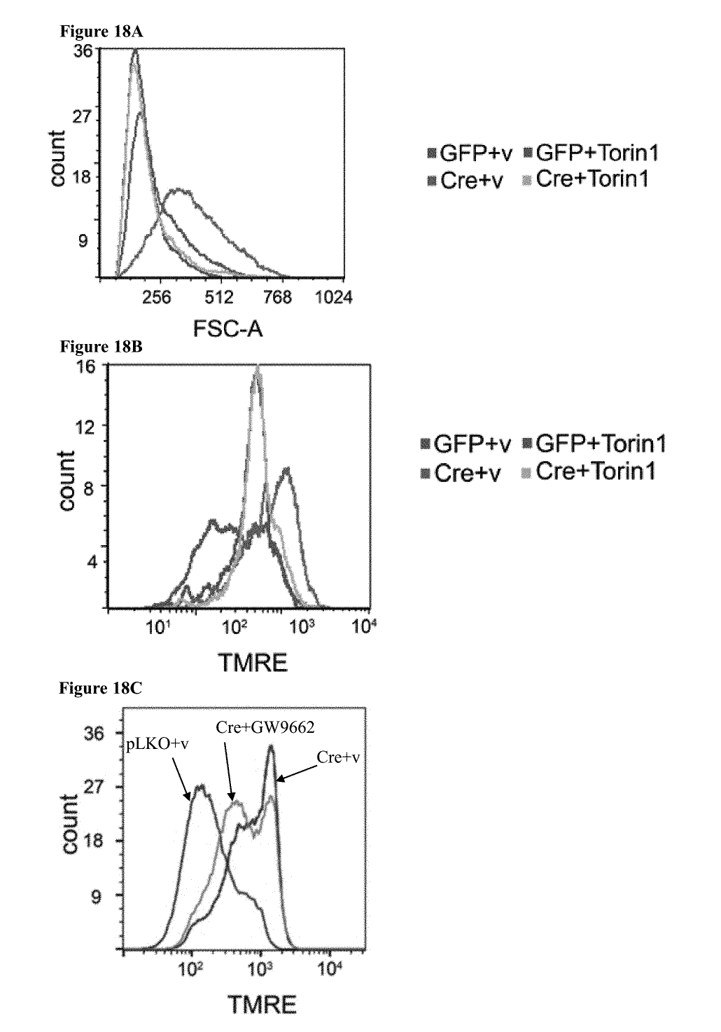

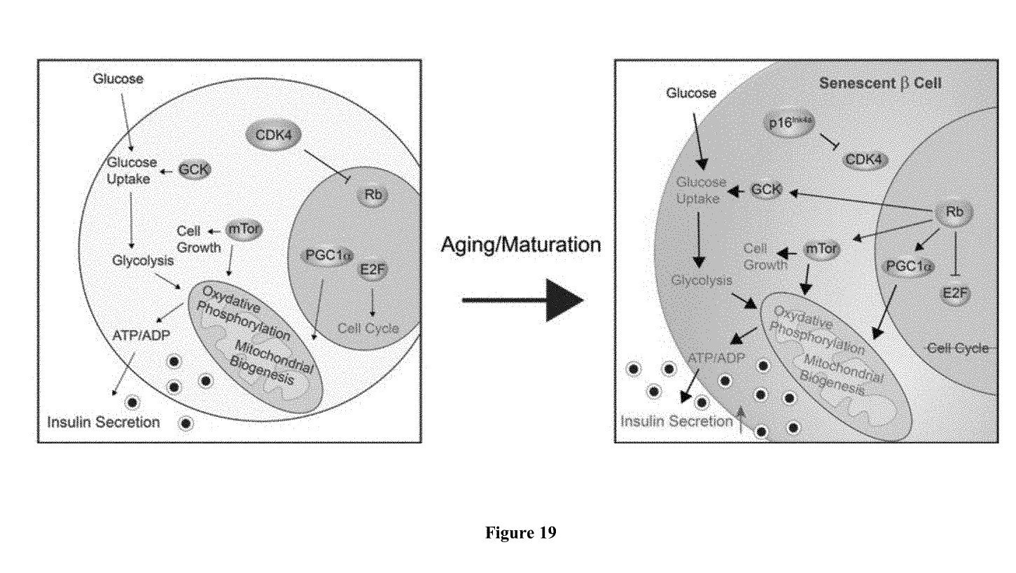

In light of the known roles of mTOR in regulating β-cell size and in mitochondrial biogenesis and function, the effects of mTOR inhibition on EndoC-βH2 were tested. Strikingly, cells treated with the kinase inhibitor Torin1 in conjunction with Cre excision exhibited lower levels of insulin secretion, as well as reduced size and mitochondrial activity (FIG. 16K and FIG. 18A-B). Inhibition of PPARγ the binding partner of Pgc1α, by treatment with of the cells with GW9662, also resulted in reduced GSIS and mitochondrial activity (FIG. 16L and FIG. 18C). These results indicate that the activity of mTOR and of the Pgc1α/PPARγ complex, which is central in the regulation of mitochondrial biogenesis and function, contributes to increased GSIS upon β-cell senescence. Together these findings indicate that p16 activity in mouse and human β-cells leads to senescence and its hallmarks—increased cell size, glucose uptake and mitochondrial activity—and to consequent increased GSIS capacity (FIG. 19).

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for enhancing insulin secretion in response to glucose in an insulin secreting cell, the method comprising:
a. providing an insulin producing cell,
b. subjecting said cell to a condition that induces or increases expression of a senescence marker, and
c. maintaining said cell in conditions such that said cell retains its identity as an insulin secreting cell,
thereby enhancing insulin secretion in an insulin secreting cell.

2. The method of claim 1, wherein said condition that induces or increases expression of a senescence marker is selected from the group consisting of: expressing a senescence inducing gene within said cell, irradiating said cell, inducing oxidative stress in said cell, and inducing DNA damage in said cell.

3. The method of claim 2, wherein said senescence inducing gene is selected from the group consisting of: p16-Ink4a, p14ARF, p53, p21 and Rb.

4. The method of claim 1, wherein said senescence marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

5. The method of claim 1, wherein said insulin secreting cell is selected from the group consisting of a beta cell and a cell engineered to secrete insulin.

6. The method of claim 1, further comprising confirming retention of an insulin secreting identity.

7. The method of claim 6, wherein said confirming comprises measuring a marker of cellular identity.

8. The method of claim 7, wherein said marker of cellular identity is selected from the group consisting of: Nkx6.1, Pdx1, Chga, Ins, Mafa, Mafb, Nkx2.2, Neurod1, Pax6, Neurog3, Slc2a2, Kcnj11, Abcc8, Ffar1, and Pcsk1.

9. The method of claim 1, further comprising confirming an increase of insulin secretion in response to glucose from said cell as compared to a control cell.

10. The method of claim 9, wherein said increase is at least a 10% increase in insulin secretion.

11. The method of claim 1, wherein said subjecting does not induce DNA damage in said cell.

12. The method of claim 11, wherein said subjecting is expressing any one of p16-Ink4a, p14ARF, p53, p21 or Rb within said cell.

13. The method of claim 11, wherein said subject suffers from a metabolic disease.

14. The method of claim 13, wherein said metabolic disease is diabetes.

15. A method of treating a metabolic disease in a subject in need thereof, the method comprising subjecting an insulin-producing cell to a condition that induces or increases expression of a senescence-associated marker and does not induce DNA damage, thereby treating a metabolic disease in said subject.

16. The method of claim 15, wherein said condition is expressing within said cell any one of p16-Ink4a, p14ARF, p53, p21 or Rb.

17. The method of claim 15, wherein said senescence marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.

18. The method of claim 15, wherein said subjecting is in-vitro or ex-vivo, the method further comprising administering the cells to the subject.

19. A composition comprising a population of modified insulin secreting cells and a carrier, said modified insulin secreting cells express one or more senescence-associated marker.

20. The composition of claim 19, wherein said senescence-associated marker is selected from the group consisting of: senescence-associated secretory phenotype, senescence-associated β-galactosidase activity, senescence-associated heterochromatin foci, chromatin alterations reinforcing senescence, p16-Ink4a, p14-ARF, p53, p21, dephosphorylated Rb, SerpinE2, Dec1, Lamp2A, HP1gamma, Ki67, BrdU incorporation, CDC47 and phosphorylated Histone H3.


SEQUENCE LISTING (9 sequences)

SEQ ID NO: 1 - DNA, 471 bp, Homo sapiens
atggagccggcggcggggagcagcatggagccttcggctgactggctggccacggccgcggcccggggtcgggtagaggaggtgcgggcgctgctggaggcgggggcgctgcccaacgcaccgaatagttacggtcggaggccgatccaggtcatgatgatgggcagcgcccgagtggcggagctgctgctgctccacggcgcggagcccaactgcgccgaccccgccactctcacccgacccgtgcacgacgctgcccgggagggcttcctggacacgctggtggtgctgcaccgggccggggcgcggctggacgtgcgcgatgcctggggccgtctgcccgtggacctggctgaggagctgggccatcgcgatgtcgcacggtacctgcgcgcggctgcggggggcaccagaggcagtaaccatgcccgcatagatgccgcggaaggtccctcagacatccccgattga

SEQ ID NO: 2 - DNA, 507 bp, Mus musculus
atggagtccgctgcagacagactggccagggcggcggcccagggccgtgtgcatgacgtgcgggcactgctggaagccggggtttcgcccaacgccccgaactctttcggtcgtaccccgattcaggtgatgatgatgggcaacgttcacgtagcagctcttctgctcaactacggtgcagattcgaactgcgaggaccccactaccttctcccgcccggtgcacgacgcagcgcgggaaggcttcctggacacgctggtggtgctgcacgggtcaggggctcggctggatgtgcgcgatgcctggggtcgcctgccgctcgacttggcccaagagcggggacatcaagacatcgtgcgatatttgcgttccgctgggtgctctttgtgttccgctgggtggtctttgtgtaccgctgggaacgtcgcccagaccgacgggcatagcttcagctcaagcacgcccagggccctggaacttcgcggccaatcccaagagcagagctaa

SEQ ID NO: 3 - protein, 156 aa, Homo sapiens
MEPAAGSSMEPSADWLATAAARGRVEEVRALLEAGALPNAPNSYGRRPIQVMMMGSARVAELLLLHGAEPNCADPATLTRPVHDAAREGFLDTLVVLHRAGARLDVRDAWGRLPVDLAEELGHRDVARYLRAAAGGTRGSNHARIDAAEGPSDIPD

SEQ ID NO: 4 - protein, 168 aa, Mus musculus
MESAADRLARAAAQGRVHDVRALLEAGVSPNAPNSFGRTPIQVMMMGNVHVAALLLNYGADSNCEDPTTFSRPVHDAAREGFLDTLVVLHGSGARLDVRDAWGRLPLDLAQERGHQDIVRYLRSAGCSLCSAGWSLCTAGNVAQTDGHSFSSSTPRALELRGQSQEQS

SEQ ID NO: 5 - DNA, 21 bp, Artificial
  Synthetic
gcagtcatagccacagcattt

SEQ ID NO: 6 - DNA, 20 bp, Artificial
  Synthetic
aagtggaaagcgaagaatcg

SEQ ID NO: 7 - DNA, 22 bp, Artificial
  Synthetic
gttacagagacggagtttggag

SEQ ID NO: 8 - DNA, 21 bp, Artificial
  Synthetic
cgtttggatgctgattatggg

SEQ ID NO: 9 - DNA, 21 bp, Artificial
  Synthetic
gcatggagccttcggctgact